(19) United States
(12) Patent Application Publication
(10) Pub. No.: US 2022/0056068 A1
(43) Pub. Date: Feb. 24, 2022

(54) LIGAND DETECTION BY APTAMERS WITH A BUILT-IN REPORTER

(71) Applicant: King Abdullah University of Science and Technology, Thuwal (SA)

(72) Inventors: Manal S. Zaher, Thuwal (SA); Fahad Rashid, Thuwal (SA); Vlad-Stefan Raducanu, Thuwal (SA); Samir M. Hamdan, Thuwal (SA)

(21) Appl. No.: 17/278,386

(22) PCT Filed: Sep. 23, 2019
(86) PCT No.: PCT/IB2019/058058

§ 371 Date: Mar. 22, 2021

Related U.S. Application Data
(60) Provisional application No. 62/734,694, filed on Sep. 21, 2018.
(60) Provisional application No. 62/774,648, filed on Dec. 3, 2018.

(51) Int. Cl. C07H 21/04; C12N 15/115; G01N 21/64

(52) U.S. Cl. CPC C07H 21/04; C12N 15/115; G01N 21/6428; G01N 2021/6432; C12N 2310/3517; G01N 2021/6439; C12N 2310/16

(57) ABSTRACT

Potassium ion sensing aptamers are disclosed. These aptamers have high specificity towards the potassium ion. Ligand-sensing aptamers with a built-in reporter are also disclosed. The built-in reporter is incorporated into the sugar-phosphate backbone of the aptamers. The built-in reporter may be an environmentally sensitive fluorescence dye, internally coupled to the aptamers. The environmentally sensitive fluorescence dye can sense the conformation changes induced by binding of the aptamers to the target ligand and transduces the conformational changes to a fluorescence change.

### CROSS-REFERENCE TO RELATED APPLICATION

The present application claims priority to U.S. Applications No. 62/734,694, filed Sep. 21, 2018 and 62/774,648 filed Dec. 3, 20198, the disclosures of which are incorporated herein by reference in their entirety.

### REFERENCE TO SEQUENCE LISTING

The Sequence Listing submitted Nov. 10, 2021 as a text file named “KAUST_028_04_ST25.txt” created on Nov. 10, 2021, and having a size of 26,814 bytes is hereby incorporated by reference.

### FIELD OF THE INVENTION

The invention is generally directed to detecting metal ion using aptamers with a built-in reporter, more particularly to detecting the potassium ion using aptamers with a built-in reporter.

### BACKGROUND OF THE INVENTION

The significant role of metal ions in biological samples and industrial applications has enticed the development of metal ion sensing technologies. In particular, potassium ion sensors have gained popularity due to their importance in analyzing the extracellular biological fluids that maintain the physiological osmolarity and transmit the neuronal signal. Potassium ion sensors are also valuable in water and soil analysis. Hence, significant effort has been directed to the development of sensors that accurately detect the potassium ion (Chen, et al. (2015) Talanta, 144, 247-251; Yang, et al. (2016) Analytical Chemistry, 88, 9285-9292).

The use of biosensors for detecting metal ions has been at the forefront of this development due to their cost effectiveness and ability to detect a wide range of ligands. However, specificity of these biosensors towards their target ligands over other mono- or divalent cations remains difficult to achieve. Additionally, they often require demanding assays and costly instrumentation (Vigneshvar, et al. (2016) Frontiers in Bioengineering and Biotechnology, 4, 11; Zhang, et al. (2011) Annual Review of Analytical Chemistry, 4, 105-128).

DNA is a rich source for designing biosensors for metal ions and other small molecules due to its versatility and ability to bind a wide range of molecular targets. In principle, two components define a sensor: a ligand recognition element and a signal transduction element (Du, et al. (2017) Analytical Chemistry, 89, 189-215; Zhang, et al. (2011) Annual Review of Analytical Chemistry, 4, 105-128). Metal ion detection technologies have witnessed a boost that stems significantly from the development of cost-effective DNA aptamers. Such metal ion sensors are valuable in both biological and industrial applications.

Aptamer-based detection relies on DNA structural changes upon ligand binding such as induction of secondary and/or tertiary structure, aggregation, and/or probe displacement. The most commonly used DNA aptamers employ the formation of G-quadruplex structure in guanine rich sequences in the presence of target metal ions such as K⁺, NH₄⁺, Na⁺, and Pb²⁺ (Ruttkay-Nedecky, et al. (2013) Molecules, 18, 14760-14779). The main limitation of these aptamer-based sensors is the requirement of an exogenous reporter that can recognize the metal-induced conformational changes and transduce them to a detectable and quantifiable signal. This limitation increases the complexity of the assays and makes them unsuitable for applications where the exogenous reporter may interfere with the ligand or the medium and compromise reproducibility. Additionally, the response of the external reporter is limited by both the dissociation constant of the ligand to the sensor and the dissociation constant of the sensor to the reporter. This additive binding effect decreases the working range as well as sensitivity of the sensor.

There is still a need for aptamers used for metal detection with improved specificity and sensitivity, for example, aptamers for detection of K⁺.

There is a need to develop aptamers with a built-in reporter for aptamer binding partner (ligand) detection, especially metal ion detection. There is also a demand to develop methods for detecting ligands, such as metal ions, using aptamers in the absence of exogenous reporters.

Therefore, it is the object of the present invention to provide aptamers with specificity to the potassium ion.

It is another object of the present invention to provide aptamers with a built-in reporter for ligand detection, especially metal ion detection.

It is another object of the present invention to provide methods for detecting ligands using aptamers with a built-in reporter.

### SUMMARY OF THE INVENTION

Disclosed are potassium ion sensing aptamers, ligand sensing aptamers with a built-in reporter, and methods of use thereof.

The potassium ion (K⁺) sensing aptamers are made of oligonucleotides, preferably single-stranded DNA molecules, preferably with a guanine (G)-rich sequence, ranging in length between 10 and 50 nt, preferably, between 14 and 30 nt, more preferably, between 18 and 22 nt, and have specificity for the potassium ion. In more preferred embodiments, the K⁺-sensing aptamers comprise, consist essentially of, or consist of the following sequences or a variant thereof.

SEQ ID NO. 1
AGGAGGGACGGGGCAGGAGGAG
SEQ ID NO. 13
GAGGGACGGGGCAGGAGG

Ligand sensing aptamers, for example, metal sensing aptamers, with a built-in reporter are also provided. Binding of the ligand sensing aptamers to the target ligand, such as a metal ion, can induce conformational changes in the aptamers, which can be sensed and transduced to a detectable and quantifiable signal by the built-in reporter. Preferred ligand-sensing aptamers are single-stranded DNA oligonucleotides with a G-rich sequence, and in some embodiments form a G-quadruplex structure in the presence of the target ligand. Particularly preferred ligand sensing aptamers with a built-in reporter are metal sensing aptamers which comprise one of the following sequences or a variant thereof,

SEQ ID NO. 26
AGGAGGGACGG/X/GGCAGGAGGAG
SEQ ID NO. 38
GAGGGACGG/X/GGCAGGAGG

where X represents a fluorescence dye as the built-in reporter.

In a preferred embodiment, the built-in reporter is incorporated into the sugar-phosphate backbone of the aptamers. The built-in reporter may be an environmentally sensitive fluorescence dye, internally coupled to the aptamers. Exemplary environmentally sensitive dyes include cyanine dyes, such as Cy3, Cy3.5, Cy5, Cy5.5, Cy7, Alexa 555, Alexa 647, and derivatives thereof.

Methods of detecting aptamer binding partners (ligands), for example, metal ions in a sample using the disclosed ligand sensing aptamers with a built-in reporter are also disclosed. The methods include (a) contacting a ligand-sensing aptamer with a built-in reporter with the sample and (b) detecting the signal transduced by the built-in reporter upon binding of the aptamer to its ligand in the sample, for example, the photophysical change of the fluorescence dye upon binding of the aptamer to the metal ion in the sample.

Additional advantages of the disclosed compounds, mixtures, compositions, kits, and methods will be set forth in part in the description which follows, and in part will be understood from the description, or may be learned by practice of the disclosed compounds, mixtures, compositions, kits, and methods. The advantages of the disclosed compounds, mixtures, compositions, kits, and methods will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed compounds, mixtures, compositions, kits, and methods, and together with the description, serve to explain the principles of the disclosed compounds, mixtures, compositions, kits, and methods.

FIG. 1 is a schematic diagram illustrating detection of metal ion (i.e., potassium) by a ligand sensing aptamer containing a built-in reporter (i.e., a cyanine dye, Cy3).

FIG. 2 is a bar graph showing the fluorescence lifetime (ns) of reporter-containing DNA oligonucleotides with different sequences in the presence of the potassium ion. The sequences of the DNA nucleotides are summarized in Table 2.

FIG. 3A is a bar graph showing the fluorescence lifetime (ns) of O-328 derivatives with different sequence lengths (grey) and their corresponding double-stranded oligonucleotides (black). FIG. 3B is a bar graph showing the fluorescence lifetime (ns) of DNA O-328 (18) (black) and RNA O-328 (18) (grey).

FIG. 4A is a graph showing the fluorescence lifetime (ns) of O-328 (18) in the presence of various concentrations of KC (log₁₀[Concentration (M)/10⁻⁹]). The experimental data were fit by Eq. (5) as described in the detailed description. FIG. 4B is a graph showing the fluorescence lifetime (ns) of an iCy5-containing DNA oligonucleotide having the same nucleotide sequence as O-328 (18) in the presence of various concentrations of KCl (log₁₀[Concentration (M)/10⁻⁹]). The experimental data were fit by Eq. (5) as described in the detailed description.

FIGS. 5A-5D show the analysis of the fluorescence lifetime response curve of O-328 (18). (A) Plot of the first (solid) and second (dotted) derivatives of the fluorescent lifetime response curve described in FIG. 4A. The analytical formulas for the derivatives are given by Eqs. (7) and (8) in the detailed description. (B) Plot of the local response coefficient as percentage, described by Eq. (10) versus dimensionless log₁₀ of the K⁺ concentration normalized to 1 nM. The value C_{R} at which the maximum response is achieved is indicated by the vertical line resided inside the interval between EC₁₀ and EC₉₀. (C) Plot of the inverse function of the response function, as described by Eq. (12). The area included in the rectangular in the middle of the graph marks the working range for the O-328 (18) sensor. (D) Plot of the theoretical relative uncertainty in the measured concentration versus dimensionless log₁₀ of the K⁺ concentration normalized to 1 nM, in the interval between EC₁₀ and EC₉₀. The plot is generated as parametric curve with the y-coordinate given by Eq. (16) and the x-coordinate given by Eq. (9) and varying the parameter t between 1.9 ns (10% response) and 2.73 ns (90% response). The concentration at which the minimum uncertainty is achieved, C_{E}, is indicated by the short vertical dashed line. The uncertainty of the lifetime measurements is indicated as Δτ=25 ps.

FIG. 6A is a graph showing the fluorescence change (%) of O-328 (18) in the presence of various concentrations of KCl (log₁₀[Concentration (M)/10⁻⁹]). The experimental data were fit by Eq. (6) as described in the detailed description. The normalized data of time-resolved fluorescence lifetime measurement was also shown in the figure for comparison. The Pearson correlation coefficient was determined between the steady-state measurement and the time-resolved measurement. FIG. 6B is a bar graph showing the absorbance change (%) of O-328 (18) in the presence of various concentrations of KCl.

FIG. 7A is a bar graph showing the fluorescence lifetime (ns) of O-328 (18) in the presence of different metal ions at 10 mM. FIG. 7B is a graph showing the fluorescence lifetime (ns) of O-328 (18) in the presence of K⁺ (♦), Na⁺ (●), or NH₄⁺ (▾) at various concentrations (log₁₀[Concentration (M)/10⁻⁹]). The experimental data were fit by Eq. (5) as described in the detailed description.

FIG. 8 shows the circular dichroism spectra of O-328 (18) in the presence of different concentrations of KCl. The inset shows the corresponding UV absorbance spectra.

FIG. 9A is a schematic diagram illustrating single molecule fluorescence measurement of O-328 (22) in the absence and presence of the potassium ion. FIG. 9B is a graph showing the iCy3 fluorescence histograms of O-328 (22) in the absence and presence of the potassium ion. The histograms were fitted to Gaussian distributions. FIG. 9C is a graph showing a representative single molecule fluorescence trace of O-328 (22), illustrating the transition to an enhanced-fluorescence state upon addition of the potassium ion. The fluorescence trace was recorded with a 100 ms temporal resolution.

FIG. 10A is a schematic diagram illustrating single molecule fluorescence measurement of O-328 (22) in the absence and presence of human replication protein A (RPA). FIG. 10B is a graph showing the iCy3 fluorescence histograms of O-328 (22) in the absence and presence of human RPA. The histograms were fitted to Gaussian distributions. FIG. 10C is a graph showing a representative single molecule fluorescence trace of O-328 (22), illustrating the transition to a reduced-fluorescence state upon addition of human RPA. The fluorescence trace was recorded with a 100 ms temporal resolution.

### DETAILED DESCRIPTION OF THE INVENTION

The disclosed compounds, mixtures, compositions, kits, and methods may be understood more readily by reference to the following detailed description of particular embodiments and the Examples included therein and to the Figures and their previous and following description.

All methods described herein can be performed in any suitable order unless otherwise indicated herein or otherwise clearly contradicted by context. The use of any and all examples or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The disclosed compounds, mixtures, compositions, and kits, can be used for, can be used in conjunction with, can be used in preparation for, or are products of the disclosed methods. It is understood that when combinations, subsets, interactions, groups, etc. of these compounds, mixtures, compositions, and kits are disclosed, while specific reference of each various individual and collective combinations of these materials may not be explicitly disclosed, each is specifically contemplated and described herein. For example, if a compound is disclosed and discussed and a number of modifications that can be made to a number of molecules including the compound are discussed, each and every combination and permutation of the compound and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the compounds, mixtures, compositions, kits, components, etc. contemplated and disclosed as above can also be specifically and independently included or excluded from any group, subgroup, list, set, etc. of such materials. These concepts apply to all aspects of this application including, but not limited to, steps in methods of making and using the disclosed compounds, compositions, mixtures, and kits. Thus, if there are a variety of additional steps that can be performed it is understood that each of these additional steps can be performed with any specific embodiment or combination of embodiments of the disclosed methods, and that each such combination is specifically contemplated and should be considered disclosed.

Throughout the description and claims of this specification, the word “comprise” and variations of the word, such as “comprising” and “comprises,” mean “including but not limited to,” and are not intended to exclude, for example, other additives, components, integers or steps.

Any discussion of documents, acts, materials, articles or the like which has been included in the present specification is not to be taken as an admission that any or all of these matters form part of the prior art base or were common general knowledge in the field relevant to the present disclosure as it existed before the priority date of each claim of this application.

### I. Definitions

The singular forms “a,” “an,” and “the” include plural reference unless the context clearly dictates otherwise. For example, reference to “a compound” includes a plurality of compounds and reference to “the compound” is a reference to one or more compounds and equivalents thereof known to those skilled in the art.

The terms “may,” “may be,” “can,” and “can be,” and related terms are intended to convey that the subject matter involved is optional (that is, the subject matter is present in some embodiments and is not present in other embodiments), not a reference to a capability of the subject matter or to a probability, unless the context clearly indicates otherwise.

The terms “optional” and “optionally” mean that the subsequently described event, circumstance, or material may or may not occur or be present, and that the description includes instances where the event, circumstance, or material occurs or is present and instances where it does not occur or is not present.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied.

Numerical ranges disclosed in the present application of any type, disclose individually each possible number that such a range could reasonably encompass, as well as any sub-ranges and combinations of sub-ranges encompassed therein.

As used herein, the term “derivatives” refers to a compound with a structure similar to that of another (reference compound) but differing from it in respect to a particular component, functional group, atom, etc. The term “derivative” also refers to a compound which is formed from a parent compound by chemical reaction(s). The differences between suitable derivatives and their reference or parent compounds include, but are not limited to, replacement of one or more chemical groups with one or more different chemical groups or reacting one or more chemical groups to introduce one or more substituents.

The term “substituents” as used herein, means replacing one or more hydrogen atoms in a chemical group or moiety independently with: a halogen atom, an alkyl group, a heteroalkyl group, an alkenyl group, a heteroalkenyl group, an alkynyl group, a heteroalkynyl group, an aryl group, a heteroaryl group, —OH, —SH, —NH₂, —N₃, —OCN, —NCO, —ONO₂, —CN, —NC, —ONO, —CONH₂, —NO, —NO₂, —ONH₂, —SCN, —SNCS, —CF₃, —CH₂CF₃, —CH₂Cl, —CHCl₂, —CH₂NH₂, —NHCOH, —CHO, —COCl, —COF, —COBr, —COOH, —SO₃H, —CH₂SO₂CH₃, —PO₃H₂, —OPO₃H₂, —P(═O)(OR^{G1′})(OR^{G2′}), —OP(═O)(OR^{G1′})(OR^{G2′}), —BR^{G1′}(OR^{G2′}), —B(OR^{G1′})(OR^{G2′}), or -G′R^{G1′} in which -G′ is —O—, —S—, —NR^{G2′}-, —C(═O)—, —S(═O)—, —SO₂—, —C(═O)O—, —C(═O)NR^{G2′}-, —OC(═O)—, —NR^{G2′}(═O)—, —OC(═O)O—, —OC(═O)NR^{G2′}-, —NR^{G2′}C(═O)O—, —NR^{G2′}C(═O)NR^{G3′}-, —C(═S)—, —C(═S)S—, —SC(═S)—, —SC(═S)S—, —C(═NR^{G2′})—, —C(═NR^{G2′})O, —C(═NR^{G2′})NR^{G3′}-, —OC(═NR^{G2′})—, —NR^{G2′}C(═NR^{G3′})—, —NR^{G2′}SO₂—, —C(═NR^{G2′})NR^{G3′}-, —OC(═NR^{G2′})—, —NR^{G2′}C(═NR^{G3′})—, —NR^{G2′}SO₂—, —NR^{G2′}SO₂NR^{G3′}-, —NR^{G2′}C(═S)—, —SC(═S)NR^{G2′}-, —NR^{G2′}C(═S)S—, —NR^{G2′}C(═S)NR^{G3′}-, —SC(═NR^{G2′})—, —C(═S)NR^{G2′}-, —OC(═S)NR^{G2′}-, —NR^{G2′}C(═S)O—, —SC(═O)NR^{G2′}-, —NR^{G2′}C(═O)S—, —C(═O)S—, —SC(═O)—, —SC(═O)S—, —C(═S)O—, —OC(═S)—, —OC(═S)O—, —SO₂NR^{G2′}-, —BR^{G2′}-, or —PR^{G2′}-; wherein each occurrence of R^{G1′}, R^{G2′}, and R^{G3′} is, independently, a hydrogen atom, a halogen atom, an alkyl group, a heteroalkyl group, an alkenyl group, a heteroalkenyl group, an alkynyl group, a heteroalkynyl group, an aryl group, or a heteroaryl group.

In some instances, the term “substituents” also refers to one or more substitutions of one or more of the carbon atoms in a carbon chain (e.g., alkyl, alkenyl, alkynyl, and aryl groups) by a heteroatom, such as, but not limited to, nitrogen, oxygen, and sulfur.

It is understood that the term “substitutions” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

“Reporter” or “sensor” as used herein refers to a self-contained analytical device that can provide information about the chemical composition of its environment. For example, it can be a chemical moiety that detects and/or measures a change in the physical property of the environment, the parent molecule, or the overall system, and records, indicates, or otherwise responds to it.

“Ligand” as used herein refers to a molecule that binds to another molecule, such as an aptamer. This term is used interchangeably with other terms such as “binding partner” and “target molecule” throughout this application. Binding between an ligand and an aptamer may occur by intermolecular forces, such as ionic bonds, hydrogen bonds and van der Waals forces.

“Aptamer” as used herein refers to single-stranded nucleic acids (DNA or RNA) that are capable of binding a specific target with high affinity and specificity. Aptamer/target binding is achieved mainly through electrostatic interactions, so the variability in aptamer sequences is what gives them their versatility. The way aptamers fold, the order of the nucleic acids, and the conditions of the matrix they are in, all contribute to binding a target. Aptamer/target binding may cause a structural switch, changing the structures and/or conformations of the aptamers, a feature that can be utilized for detection methods.

“Oligonucleotide” refers to short nucleic acid (i.e., DNA and RNA) molecules. They contain 100 or less nucleotides. Preferably, they contain 50 or less nucleotides. More preferably, they contain 25 or less nucleotides. Most preferably, they contain between 14 and 22, inclusive, nucleotides.

“Environmentally sensitive fluorescence dye” refers to fluorescence dyes having photophysical properties that are responsive to physicochemical changes in the local environment including pH, viscosity, biological or non-biological analytes, and solvent polarity. The photophysical properties may include the fluorescence wavelength, the fluorescence intensity, fluorescence life time, and combinations thereof. For example, the fluorescence intensities of certain dyes may be highly sensitive to the polarity of their local environments; their fluorescence signals may be more intense and blue-shifted when they are in a less polar environment.

“Carrier” refers to all components present in a formulation or composition other than the active ingredient or ingredients. They may include but are not limited to diluents, binders, lubricants, desintegrators, fillers, plasticizers, pigments, colorants, stabilizing agents, and glidants.

### II. Potassium Ion Sensing Aptamers

Disclosed are potassium ion sensing aptamers. These aptamers have high specificity for the potassium ion. Preferably, they have higher specificity for the potassium ion than other metal ions, such as sodium and/or magnesium.

In certain embodiments, the potassium ion sensing aptamers comprise, consist essentially of, or consist of one of the following sequences:

SEQ ID NO. 1
AGGAGGGACGGGGCAGGAGGAG
SEQ ID NO. 2
GGAGGGACGGGGCAGGAGGAG
SEQ ID NO. 3
AGGAGGGACGGGGCAGGAGGA
SEQ ID NO. 4
GAGGGACGGGGCAGGAGGAG
SEQ ID NO. 5
GGAGGGACGGGGCAGGAGGA
SEQ ID NO. 6
AGGAGGGACGGGGCAGGAGG
SEQ ID NO. 7
AGGGACGGGGCAGGAGGAG
SEQ ID NO. 8
GAGGGACGGGGCAGGAGGA
SEQ ID NO. 9
GGAGGGACGGGGCAGGAGG
SEQ ID NO. 10
AGGAGGGACGGGGCAGGAG
SEQ ID NO. 11
GGGACGGGGCAGGAGGAG
SEQ ID NO. 12
AGGGACGGGGCAGGAGGA
SEQ ID NO. 13
GAGGGACGGGGCAGGAGG
SEQ ID NO. 14
GGAGGGACGGGGCAGGAG
SEQ ID NO. 15
AGGAGGGACGGGGCAGGA
SEQ ID NO. 16
GGGACGGGGCAGGAGGA
SEQ ID NO. 17
AGGGACGGGGCAGGAGG
SEQ ID NO. 18
GAGGGACGGGGCAGGAG
SEQ ID NO. 19
GGAGGGACGGGGCAGGA
SEQ ID NO. 20
GGGACGGGGCAGGAGG
SEQ ID NO. 21
AGGGACGGGGCAGGAG
SEQ ID NO. 22
GAGGGACGGGGCAGGA
SEQ ID NO. 23
GGGACGGGGCAGGAG
SEQ ID NO. 24
AGGGACGGGGCAGGA
SEQ ID NO. 25
GGGACGGGGCAGGA

In certain embodiments, the potassium ion sensing aptamers have between 14 and 22, inclusive, nucleotides.

The potassium ion sensing aptamers can be used to detect the presence of potassium ions in a sample, preferably using the built-in sensor design disclosed herein.

### III. Ligand Sensing Aptamers with a Built-In Reporter

Ligand sensing aptamers, for example, metal sensing aptamers, with a built-in reporter are provided. While not being bound by theory, binding of these aptamers to the target ligand, such as a metal ion can induce conformations changes, which can be sensed and transduced to a detectable and quantifiable signal by the built-in reporter.

1. Aptamer Sequence

The ligand sensing aptamers with a built-in reporter may contain sequences as single-stranded oligonucleotides, preferably rich in guanine nucleotides. While not being bound by theory, formation of G-quadruplex structure may occur in the guanine-rich sequences in the presence of the target ligand, which can be sensed by the built-in reporter and can be transmitted to a dateable signal.

For example, the oligonucleotides may have a sequence in which at least 40, 45, 50, 55, 60, 65, or 70 percent of the nucleotides are guanine nucleotides.

In some embodiments, the oligonucleotides have 100 or less nucleotides, 50 or less nucleotides, or 25 or less nucleotides. Preferably, the oligonucleotides have between 14 and 22, inclusive, nucleotides. For example, the oligonucleotides may contain 14, 15, 16, 17, 18, 19, 20, 21, or 22 nucleotides.

In some embodiments, the oligonucleotides are DNA oligonucleotides.

In some embodiments, the oligonucleotides contain one of the following sequences:

SEQ ID NO. 26
AGGAGGGACGG/X/GGCAGGAGGAG
SEQ ID NO. 27
GGAGGGACGG/X/GGCAGGAGGAG
SEQ ID NO. 28
AGGAGGGACGG/X/GGCAGGAGGA
SEQ ID NO. 29
GAGGGACGG/X/GGCAGGAGGAG
SEQ ID NO. 30
GGAGGGACGG/X/GGCAGGAGGA
SEQ ID NO. 31
AGGAGGGACGG/X/GGCAGGAGG
SEQ ID NO. 32
AGGGACGG/X/GGCAGGAGGAG
SEQ ID NO. 33
GAGGGACGG/X/GGCAGGAGGA
SEQ ID NO. 34
GGAGGGACGG/X/GGCAGGAGG
SEQ ID NO. 35
AGGAGGGACGG/X/GGCAGGAG
SEQ ID NO. 36
GGGACGG/X/GGCAGGAGGAG
SEQ ID NO. 37
AGGGACGG/X/GGCAGGAGGA
SEQ ID NO. 38
GAGGGACGG/X/GGCAGGAGG
SEQ ID NO. 39
GGAGGGACGG/X/GGCAGGAG
SEQ ID NO. 40
AGGAGGGACGG/X/GGCAGGA
SEQ ID NO. 41
GGGACGG/X/GGCAGGAGGA
SEQ ID NO. 42
AGGGACGG/X/GGCAGGAGG
SEQ ID NO. 43
GAGGGACGG/X/GGCAGGAG
SEQ ID NO. 44
GGAGGGACGG/X/GGCAGGA
SEQ ID NO. 45
GGGACGG/X/GGCAGGAGG
SEQ ID NO. 46
AGGGACGG/X/GGCAGGAG
SEQ ID NO. 47
GAGGGACGG/X/GGCAGGA
SEQ ID NO. 48
GGGACGG/X/GGCAGGAG
SEQ ID NO. 49
AGGGACGG/X/GGCAGGA
SEQ ID NO. 50
GGGACGG/X/GGCAGGA

wherein X represents a fluorescence dye as the built-in reporter.

Additional sequences which can be used to make ligand sensing aptamers with a built-in reporter are the sequences of G-quadruplex forming aptamers, which are known in the art. DNA G-quadruplex forming aptamers are reviewed in Tucker, et al, Current Pharmaceutical Design, 2012, 18, 2014-2026. Exemplary sequences and their targets are listed in Table 1 below. See also Kwok, et al., Trends in Biotechnol., 35(10):997-1013 (2017).

TABLE 1
DNA G-quadruplex Aptamers: Targets and Sequences

| Target | Sequence (5′ to 3′) |
|---|---|
| Thrombin | d(GGTTGGTGTGGTTGG) |
| | (SEQ ID NO: 51) |
| Nucleolin | d(GGTGGTGGTGGTTGTGGTGGTGGTGG) |
| | (SEQ ID NO: 52) |
| VEGF | d(TGTGGGGGTGGACGGGCCGGGTAGA) |
| | (SEQ ID NO: 53) |
| | d(TGTGGGGGTGGACTGGGTGGGTACC) |
| | (SEQ ID NO: 54) |
| HIV-1 gp120 | d(TTGGGTT) |
| V3 loop | (SEQ ID NO: 55) |
| HIV-1 reverse | d(GGGGGTGGGAGGGTAGGCCTTAGGTTTCTGA) |
| transcriptase | (SEQ ID NO: 56) |
| Insulin | d(GGTGGTGGGGGGGGTTGGTAGGGTGTCTTC) |
| | (SEQ ID NO: 57) |
| Potassium | d(GGGTTAGGGTTAGGGTAGGG) |
| ions | (SEQ ID NO: 58) |
| ATP | d(CCTGGGGGAGTATTGCGGAGGAAGG |
| | (SEQ ID NO: 59) |

The ligand sensing aptamers with a built-in reporter can also contain oligonucleotide sequences of RNA G-quadruplex based aptamers. RNA G-quadruplex based aptamers are known in the art. See, for example, Agarwala, et al., Org. Biomol. Chem., 2015, 13, 5570-5585. Exemplary sequences include r(GGA)₄ (SEQ ID NO. 60 and r(GGA)₂ (SEQ ID NO. 61 (see Medic, et al., Croat. Chem. Acta, 87(4):321-325 (2014)).

Representative examples of how to make and use aptamers to bind a variety of different target molecules can be found in U.S. Pat. Nos. 5,476,766, 5,503,978, 5,631,146, 5,731,424, 5,780.228, 5,792,613, 5,795,721, 5,846,713, 5,858,660, 5,861,254, 5,864,026, 5,869,641, 5,958,691, 6,001,988, 6,011,020, 6,013,443, 6,020, 130, 6,028,186, 6,030,776, and 6,051,698. For example, aptamers with affinity for a desired metal ion can be selected from a large oligonucleotide library through Sequential Evolution of Ligands by Exponential Enrichment (SELEX). Through an iterative process, non-binding aptamers are discarded and aptamers binding to the proposed target are expanded. Initial positive selection rounds are sometimes followed by negative selection. This improves the selectivity of the resulting aptamer candidates. Multiple rounds of SELEX can be performed with increasing stringency to enhance enrichment of the oligonucleotide pool. Detailed principles and approaches used in SELEX can be found in Darmostuk, et al. (2015) Biotechnology Advances, 33, 1141-1161; Wu, et al. Methods, 106, 21-28; and references cited therein.

2. Built-In Reporter

The built-in reporter is covalently incorporated in the ligand sensing aptamers. Preferably, the built-in reporter is incorporated in the sugar-phosphate backbone of the oligonucleotide sequence of the ligand sensing aptamers via covalent linkages with the neighboring nucleotides, rather than post-synthetic labeling via N-hydroxysuccinimide (NHS)-amine chemistry. This approach provides the reporter with greater sensitivity to conformational changes of the aptamers, compared to post-synthetic labeling. Accordingly, in preferred embodiments, the built-in reporter is not incorporated into the aptamer using NHS-amine chemistry, for example, the built-in reporter is not linked to the aptamer by an amide bond. The built-reporter can transduce the conformational changes of the ligand sensing aptamers to a detectable and quantifiable signal. Preferably, it does not interfere with the binding between the aptamers and the target ligand or the conformational changes induced by binding of the aptamers to the target ligand.

i. Fluorescence Dye

In some embodiments, the built-in reporter is a fluorescence dye. Conformational changes of the ligand sensing aptamers may induce a photophysical change of the fluorescence dye.

In some embodiments, the fluorescence dye is an environmentally sensitive fluorescence dye, such as Cy3, Cy3.5, Cy5, Cy5.5, Cy7, Cy7.5, Alexa 555, Alexa 647, DyLight 547, DyLight 548, DyLight 549, and DyLight 647. These fluorescence dyes may be in different forms, for example, sulfonated or non-sulfonated, and may be incorporated in the backbone of sugar-phosphate backbone of the oligonucleotide sequence of the ligand sensing aptamers via different strategies, such as phosphoramidite chemistry and Click Chemistry.

In some embodiments, the environmentally sensitive fluorescence dye is a cyanine dye as described in U.S. Pat. Nos. U.S. Pat. Nos. 6,225,050 and 6,956,032; Discovery of New Fluorescent Dyes: Targeted Synthesis or Combinatorial Approach?, in Advanced Fluorescence Reporters in Chemistry and Biology I, Springer Berlin Heidelberg, 2010; Fluorescence-Based Biosensors, in Progress in Molecular Biology and Translational Science, Elsevier, 2013; and references cited therein.

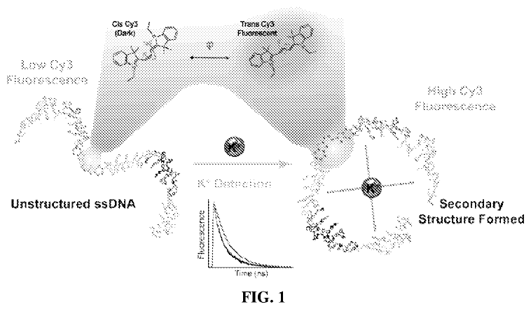

The fluorescence properties of cyanine dyes such as Cy3 and Cy5 depend on the structural environment of the coupled DNA-dye complex (Levitus, et al. (2011) Quarterly Reviews of Biophysics, 44, 123-151; Sanborn, et al. (2007) Journal of Physical Chemistry B, 111, 11064-11074). The cyanine dyes exhibit interesting photophysical properties due to their non-rigid structure with a polymethine bond connecting two nitrogen-containing chemical moieties. The fluorescence of these dyes as quantified by fluorescence intensity, fluorescence lifetime or quantum yield is dependent on the cis-trans photoisomerization rate around the polymethine bond, as illustrated in FIG. 1. This photoisomeriztion rate in turn hinges on the overall environment of the fluorophore (Levitus, et al. (2011) Quarterly Reviews of Biophysics, 44, 123-151).

Exemplary cyanine dyes include Cy3, Cy3.5, Cy5, Cy5.5, Cy7, Cy7.5, and derivatives thereof.

Cy3 may have a structure of the following formula or a derivative thereof:

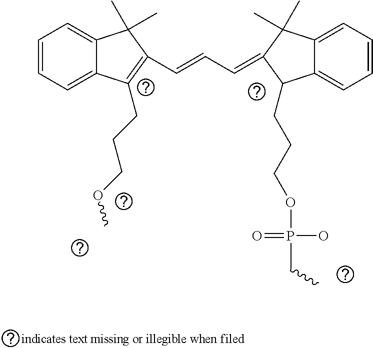

Cy5 may have a structure of the following formula or a derivative thereof:

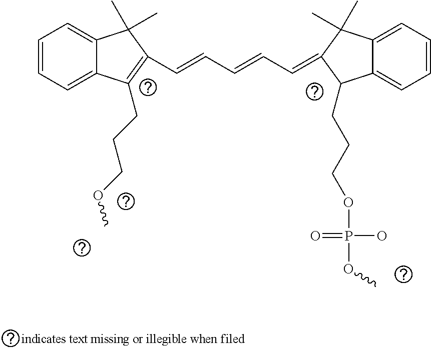

In some embodiments, the photophysical change of the fluorescence dye is a shift in the fluorescence wavelength, a change in the fluorescence signal intensity, a change in the fluorescence life time, or combinations thereof. The fluorescence dye may be non-fluorescent or fluorescent-quenched prior to binding of the metal ion and may become fluorescent or fluorescent-enhanced upon binding of the target ligand, such as a metal ion.

3. Ligand Detection

The ligand sensing aptamers are engineered to detect their binding partners (i.e., ligands) in a sample. Preferred ligands are metal ions. In some embodiments, the metal ion under detection is selected from potassium, lithium, magnesium, manganese, calcium, cesium, zinc, sodium, potassium, and strontium. In some embodiments, the metal ion under detection is potassium. In some embodiments, the aptamers can be engineered to detect non-metal cations, such as ammonium.

Binding between the aptamers and its target ligand is achieved mainly through electrostatic interactions. Binding of the target ligand, such as a metal ion, can induce a conformational change of the aptamers. A specific secondary structure of the aptamers may be formed upon metal binding. For the G-rich aptamers, the ligand-induced secondary structure may be similar to G-quadruplexes as described in Campbell, et al. (2012) Metal Ions in Life Sciences, 10, 119-134.

FIG. 1 illustrates an exemplary scheme on detection of metal ion using a metal sensing aptamer containing a cyanine dye, Cy3. Cy3 is an environmentally sensitive fluorophore capable of photoisomerizing between a cis and a trans conformer owing to its flexible polymethine bond. Cy3 fluorescence increase stems from a decrease in its photoisomerization rate induced by the structural rearrangement of the aptamer. Binding of the metal sensing aptamer to the target ligand (e.g., the potassium ion) induces formation of a secondary structure. The overall structural change of the aptamer decrease the Cy3 photoisomerization rate, leading to an increase in the fluorescence signal and lifetime.

### IV. Mixtures, Compositions, and Kits

Disclosed are mixtures and compositions formed by performing or preparing to perform the disclosed methods.

For example, disclosed are mixtures containing multiple aptamers. The aptamers in the mixtures may have different specificity towards different ligands. The aptamers in the mixtures may contain different built-in reporters.

In another example, disclosed are compositions containing one or more aptamers as well as one or more other compounds, solvents, or materials. The compositions may be in the form of solutions, suspensions, emulsions, powders, and solid cakes.

The compounds, mixtures, and compositions described above can be packaged together with other components in any suitable combination as a kit useful for performing, or aiding in the performance of, the disclosed methods. It is useful if the components in a given kit are designed and adapted for use together in the disclosed methods.

In one aspect disclosed are kits for detecting ligands, such as metal ions, in a sample. The kits contains, in one or more containers, one or more of the disclosed aptamers, mixtures, and compositions as well as one or more other components, such as compounds, solvents, and materials, as carriers. The carriers do not interfere with the effectiveness of the disclosed aptamers in detecting the ligands. The kits may also include instructions to use.

### V. Methods of Making

The disclosed aptamers can be readily synthesized using techniques generally known to biochemists and synthetic chemists. An exemplary method to synthesize the aptamers is via phosphoramidite chemistry as illustrated in Itakura et al. (1984) Ann. Rev. Biochem., 53, 323-56.

The built-in reporter can be incorporated in the sequence of the metal sensing aptamer sequence via covalent linkages to its neighboring nucleotides. Preferably, the covalent linkages are formed between the built-in reporter and the five-carbon sugar or phosphate group of the neighboring nucleotides. Preferably, the covalent linkages are formed via phosphoramidite chemistry.

For example, the built-in reporter can be covalently linked to the five-carbon sugar (through the 3-OH group on the five-carbon sugar) of its neighboring nucleotide from the 5′ end and to the phosphate group of its neighboring nucleotide from the 3′ end. A representative example is shown below, in which a Cy3 dye is incorporated in the sugar-phosphate backbone of the sequence of an oligonucleotide.

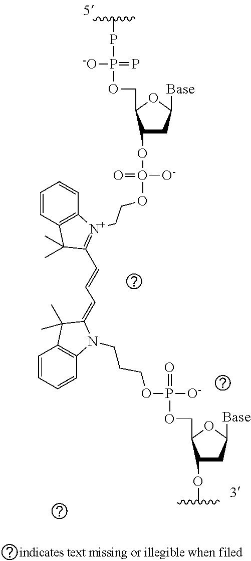

### VI. Methods of Use

Disclosed are methods for detecting target molecules (i.e., ligands) using the disclosed ligand sensing aptamers with a built-in reporter. The methods include (a) contacting a sample containing the target molecules with one or more of the disclosed aptamers and (b) monitoring signal change from the built-in reporter. Detection of the signal change from the built-in reporter indicates conformational changes of the aptamers induced by binding to the target molecules.

The sample containing the target molecules may be biological or non-biological samples. In certain embodiments, the sample may be or contain a human or non-human animal bodily fluid, a human or non-human animal tissue, or both. Exemplary bodily fluids include saliva, sputum, blood serum, blood, urine, mucus, vaginal lubrication, pus, cerebrospinal fluid, and wound exudate.

The ligand sensing aptamers may be metal sensing aptamers that are single-stranded oligonucleotides, having a sequence in which at least 40, 45, 50, 55, 60, 65, or 70 percent of the nucleotides are guanine nucleotides and a fluorescence dye that is incorporated in the sugar-phosphate backbone of the sequence of the oligonucleotide via covalent linkages with its neighboring nucleotides. The methods for detecting metal ion may include (a) contacting a sample containing the metal ion with one or more of the disclosed oligonucleotides and (b) detecting photophysical change of the fluorescence dye of the oligonucleotides. Detection of photophysical change of the fluorescence dye indicates conformational changes of the oligonucleotides induced by binding to the metal ion.

In some embodiments, the oligonucleotides have between 14 and 22, inclusive, nucleotides. In some embodiments, the oligonucleotides are DNA oligonucleotides. In some embodiments, the oligonucleotides contain or have a sequence from SEQ ID NO. 26-50.

In some embodiments, the fluorescence dye of the oligonucleotides is an environmentally sensitive fluorescence dye. In some embodiments, the environmentally sensitive fluorescence dye is a cyanine dyes. Exemplary cyanine dyes include Cy3, Cy3B, Cy3.5, Cy5, Cy5.5, Cy7, Cy7.5, and derivatives thereof.

The disclosed methods can be used to detect a wide range of metal ions. In some embodiments, the metal ion under detection is selected from potassium, lithium, magnesium, manganese, calcium, cesium, zinc, sodium, potassium, and strontium. In some embodiments, the metal ion under detection is potassium. In some embodiments, the methods can also be used to detect non-metal cations, such as ammonium.

In some embodiments, the photophysical change of the fluorescence dye is a shift in the fluorescence wavelength, a change in the fluorescence signal intensity, a change in the fluorescence life time, or combinations thereof. The fluorescence dye may be non-fluorescent or fluorescent-quenched prior to binding of the metal ion and may become fluorescent or fluorescent-enhanced upon binding of the metal ion.

Fluorescence measurement can be performed at a steady-state and/or time-resolved setup. Steady-state fluorescence measurement can be advantageous by the use of microplate-readers, which allow high-throughput screening of many different conditions. Time-resolved fluorescence measurements offer better reproducibility and minimization of error.

The disclosed methods also include combinational use of multiple aptamers. The aptamers may be combined to form mixtures or compositions as described previously.

The aptamers in the mixtures or compositions may have different specificity towards different ligands, such as different metal ions, allowing for the coverage of a wide range of metal ions. In some embodiments, the aptamers in the mixtures or combinations may contain different built-in reporters so that each kind of metal ion can be selectively recognized or detected.

### Examples

Unless otherwise stated, oligonucleotides in the Examples refer to single-stranded (ss) DNA oligonucleotides. All salts used in the Examples are HPLC grade and were purchased from Sigma Aldrich. DNase-free water was used to dissolve salts and/or DNA oligonucleotides.

### Example 1. DNA Oligonucleotides Containing a Cyanine Dye

DNA oligonucleotides containing a Cy3 dye (i.e., iCy3) or a Cy5 dye (i.e., iCy5) were purchased from Integrated DNA technologies (IDT), Inc. The chemical structure of iCy3 and iCy5 in the oligonucleotides are shown below. They are incorporated in the sequences of the oligonucleotides via phosphoramidite chemistry.

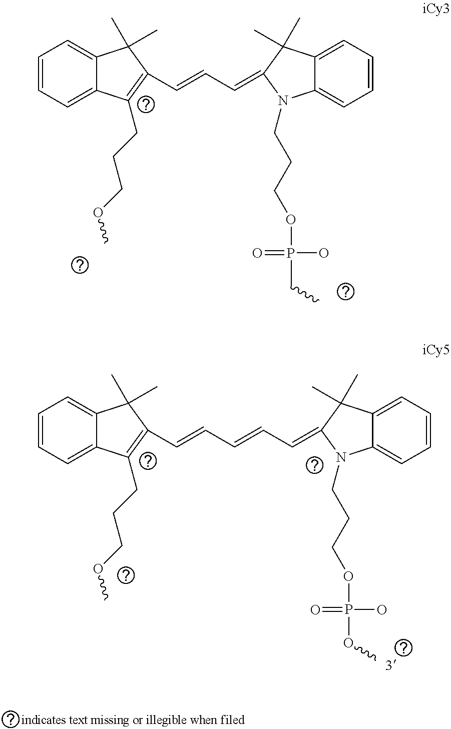

### Example 2. Fluorescence Lifetime Measurements of iCy3-Labeled DNA Oligonucleotides

Materials and Methods

Time-resolved fluorescence lifetime measurements of DNA oligonucleotides labeled with iCy3 were performed in a buffer containing 50 mM HEPES, pH 7.5, 50 mM KCl, 5% glycerol, and 1 mM MgCl₂. The final concentration of the DNA oligonucleotides with iCy3 was 50 nM.

Table 2 summarizes the sequences of the iCy3-labeled DNA oligonucleotides. The list includes 46 DNA oligonucleotides labeled with iCy3 at the 5′ end, at the 3′ end, or within the sequence (i.e., internally labeled).

TABLE 2
Sequences of iCy3-labeled DNA oligonucleotides

| | |
|---|---|
| Oligo Name | Oligo Sequence |

5′-iCy3-labeled library

| | |
|---|---|
| O-313 | /5Cy3/AAAAAAAAAAAAAAAAAAAAA |
| | (SEQ ID NO: 62) |
| O-316 | /5Cy3/CTCTCTCTCTCTCTCTCTCTC |
| | (SEQ ID NO: 63) |
| O-319 | /5Cy3/TTTTTTTTTTTTTTTTTTTTT |
| | (SEQ ID NO: 64) |
| O-320 | /5Cy3/CCCCCCCCCCCCCCCCCCCCC |
| | (SEQ ID NO: 65) |
| O-270 | /5Cy3/TGAATGAATGACTGCCTGACT |
| | (SEQ ID NO: 66) |
| O-297 | /5Cy3/GAAAAAGTTAGGACTGCTCGTCATC |
| | (SEQ ID NO: 67) |
| O-287 | /5Cy3/GAAAAGAATGACTGCCTGACT |
| | (SEQ ID NO: 68) |
| O-318 | /5Cy3/AAGAAGAGTTTACTGTGAAGA |
| | (SEQ ID NO: 69) |
| O-290 | /5Cy3/CCCCCGAATGACTGCCTGACT |
| | (SEQ ID NO: 70) |
| O-289 | /5Cy3/TGCGCGAATGACTGCCTGACT |
| | (SEQ ID NO: 71) |
| O-312 | /5Cy3/TAAGTTAGGACTGCTCGTCAT |
| | (SEQ ID NO: 72) |
| O-110 | /5Cy3/TAAGTTAGGACTGCTCGTCATC |
| | (SEQ ID NO: 73) |
| O-298 | /5Cy3/CCCTTAGTTAGGACTGCTCGTCATC |
| | (SEQ ID NO: 74) |
| O-317 | /5Cy3/TTCTTCAGTTCAGCCATCCAT |
| | (SEQ ID NO: 75) |
| O-364 | /5Cy3/GGGAAGCCCGGTGCCGAGAAG |
| | (SEQ ID NO: 76) |
| O-314 | /5Cy3/CTCTTCAGTTCAGCCATCTCA |
| | (SEQ ID NO: 77) |
| O-291 | /5Cy3/CCCTTGAATGACTGCCTGACT |
| | (SEQ ID NO: 78) |
| O-288 | /5Cy3/GAGAAGAATGACTGCCTGACT |
| | (SEQ ID NO: 79) |
| O-367 | /5Cy3/GGGGGGGGGGACTGACTGACT |
| | (SEQ ID NO: 80) |

3′-iCy3-labeled library

| | |
|---|---|
| O-331 | AAAAAAAAAAAAAAAAAAAAA/3Cy3/ |
| | (SEQ ID NO: 81) |
| O-306 | TGAATGAATGACTGCCTTCCC/3Cy3/ |
| | (SEQ ID NO: 82) |
| O-305 | TGAATGAATGACTGCCAATAG/3Cy3/ |
| | (SEQ ID NO: 83) |
| O-334 | CTCTCTCTCTCTCTCTCTCTC/3Cy3/ |
| | (SEQ ID NO: 84) |
| O-302 | TGAATGAATGACTGCCAAAAG/3Cy3/ |
| | (SEQ ID NO: 85) |
| O-304 | TGAATGAATGACTGCCCCCCC/3Cy3/ |
| | (SEQ ID NO: 86) |
| O-338 | CCCCCCCCCCCCCCCCCCCCC/3Cy3/ |
| | (SEQ ID NO: 87) |
| O-337 | TTTTTTTTTTTTTTTTTTTTT/3Cy3/ |
| | (SEQ ID NO: 88) |
| O-275 | TGAATGAATGACTGCCTGACT/3Cy3/ |
| | (SEQ ID NO: 89) |
| O-335 | TTCTTCAGTTCAGCCATCCAT/3Cy3/ |
| | (SEQ ID NO: 90) |
| O-333 | GAGAAGCCCGGTGCCGAGAAG/3Cy3/ |
| | (SEQ ID NO: 91) |
| O-303 | TGAATGAATGACTGCCAAGAG/3Cy3/ |
| | (SEQ ID NO: 92) |
| O-366 | ACTGACTGACTGGGGGGGGGG/3Cy3/ |
| | (SEQ ID NO: 93) |
| O-336 | AAGAAGAGTTTACTGTGAAGA/3Cy3/ |
| | (SEQ ID NO: 94) |
| O-330 | TAAGTTAGGACTGCTCGTCAT/3Cy3/ |
| | (SEQ ID NO: 95) |

Internal iCy3-labeled library

| | |
|---|---|
| O-327 | AAAAAAAAAAA/iCy3/AAAAAAAAAAA |
| | (SEQ ID NO: 96) |
| O-325 | TTTTTTTTTTT/iCy3/TTTTTTTTTTT |
| | (SEQ ID NO: 97) |
| O-389 | CCCTGATAA/ICY3/AAAAGCCTACC |
| | (SEQ ID NO: 98) |
| O-271 | TGAA/iCy3/GAATGACTGCCTGACT |
| | (SEQ ID NO: 99) |
| O-326 | CCCCCCCCCCC/iCy3/CCCCCCCCCCC |
| | (SEQ ID NO: 100) |
| O-272 | TGAATGAA/iCy3/GACTGCCTGACT |
| | (SEQ ID NO: 101) |
| O-393 | GAAAACC/iCy3/GTACTTCCAATCC |
| | (SEQ ID NO: 102) |
| O-273 | TGAATGAATGAC/iCy3/GCCTGACT |
| | (SEQ ID NO: 103) |
| O-390 | ACCGAAGGAACAT/iCy3/TCGTCCG |
| | (SEQ ID NO: 104) |
| O-387 | TTGATGAGCCCA/ICY3/GGAAGTTG |
| | (SEQ ID NO: 105) |
| O-274 | TGAATGAATGACTGCC/iCy3/GACT |
| | (SEQ ID NO: 106) |
| O-328 | AGGAGGGACGG/iCy3/GGCAGGAGGAG |
| | (SEQ ID NO: 107) |

The fluorescence lifetime measurements were performed in the time-correlated single-photon counting (TCSPC) mode using a QuantaMaster 800 spectrofluorometer (Photon Technology International Inc.) equipped with a Fianuim supercontinuum fiber laser source with 6 ps pulse width, operated at 20 MHz repetition rate. Data were recorded at room temperature under magic angle (54.7°) and photons were counted using a time-to-amplitude converter (TAC) and a Becker-Hickl single photon counting card. To reduce the collection of scattered light, a longpass filter (550 nm) was placed at the emission side. In all measurements, 10,000 counts were acquired. The instrument response function (IRF) was estimated using a Ludox colloidal silica suspension dissolved in water. The samples were excited at 535 nm (λₘₐₓ₋ₑₓ of iCy3) and emission was collected at 565 nm (λₘₐₓ₋ₑₘ of iCy3) with 5 nm slit width for both excitation and emission.

For a mixture of two populations with two different fluorescence lifetimes and same absorption and emission spectra, the measured lifetime is a linear combination of the concentration-weighted individual lifetimes (Palo, et al. (2002) Biophysical Journal, 83, 605-618). The amplitude-averaged lifetimes of iCy3 were estimated by fitting lifetime decays to a double-exponential equation using the FluoFit software package (PicoQuant) and applying the IRF. The best fit was chosen based on reduced chi-square and randomness of the residuals. The fitting yielded an amplitude-weighted average of fluorescence lifetimes.

Results

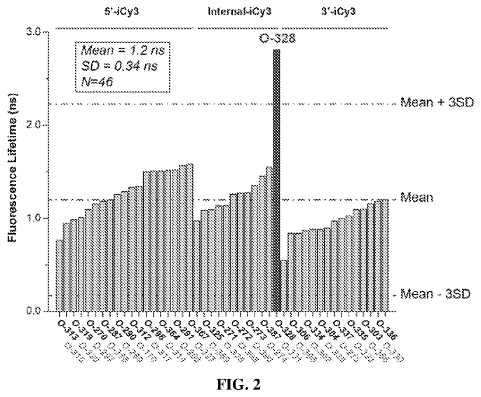

In the presence of 50 mM KCl, the O-328 (22) oligonucleotide containing an internal iCy3 (sequence: AGGAGGGACGG/iCy3/GGCAGGAGGAG) (SEQ ID NO:107) exhibited the longest fluorescence lifetime among the 46 DNA oligonucleotides labeled with iCy3 (FIG. 2). Thus, it is evident that the sequence of O-328 is highly specific for K⁺.

### Example 3. Fluorescence Lifetime Measurements of O-328 Derivatives

Materials and Methods

Time-resolved fluorescence lifetime measurements of O-328 derivatives containing an internal iCy3 were performed in a buffer containing 50 mM HEPES-KOH, pH 7.5, 50 mM KCl, 5% glycerol, and 1 mM MgCl₂. The final concentration of O-328 derivatives was 50 nM. Data were recorded as described in Example 2.

Results

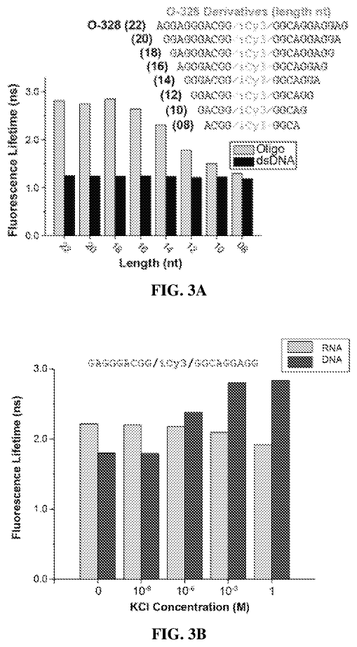

FIG. 3A shows the effect of sequence length on the fluorescence lifetime of a set of O-328 derivatives. O-328 (18) exhibited the longest fluorescence lifetime among all the tested O-328 derivatives. The fluorescence lifetime of the O-328 derivatives remained largely consistent (˜2.75 ns), up to a length of 16 nt; then it dropped down gradually as the sequence length decreased to 8 nt (1.3 ns). Upon annealing the corresponding complementary strand, the formed double-stranded (ds) DNA oligonucleotides exhibited a constant lifetime (˜1.2 ns) across different lengths (FIG. 3A). These results indicate that a specific secondary structure was present within the core of O-328 upon binding of the potassium ion. Annealing the corresponding complementary strands to form ds oligonucleotides, however, prevented the formation of this secondary structure.

FIG. 3B shows the fluorescence lifetime of DNA O-328 (18) and RNA O-328 (18) in the presence of different concentrations of KCl. The fluorescence lifetime of DNA O-328 (18) increased as the concentration of KCl increased, whereas the fluorescence lifetime of RNA O-328 (18) was insensitive to the addition of KCl.

Taken together, it is evident that simple modifications such as shortening the sequence length, converting ss DNA to ds DNA, or switching from deoxyribose to ribose can abolish the ability of O-328 in sensing the potassium ion.

### Example 4. Theoretical Formulas for Fitting the Metal Ion Concentration Dependence of Fluorescence Properties

Considering a system with two states of a receptor, P₀ and P_{∞}, and that the transition between the two states is induced by binding of a ligand to this receptor. In the case when [P₀]<<[L₀] (i.e., the ligand is at least 10 fold in excess compared to the receptor, giving ([L₀]−[PL])=[L]≈[L₀], since [PL]≤[P₀]<<[L₀]), the binding isotherm is described by a Hill function (Gesztelyi, et al. (2012) Archive for History of Exact Sciences, 66, 427-438 and Stefan, et al. (2013) PLoS Computational Biology, 9, e1003106), which gives the fraction of receptor that is bound to the ligand at any initial ligand concentration, as follows:

$$w ( [ {L}_{0} ] ) = \frac{1}{1 + {( \frac{{K}_{D}}{[ L ]} )}^{n}} \approx \frac{1}{1 + {( \frac{{K}_{D}}{[ {L}_{0} ]} )}^{n}} \qquad ( 1 )$$

where K_{D} is the microscopic dissociation constant, n is the Hill coefficient, [L₀] is the total ligand concentration, [L] is the free ligand concentration, [P₀] is the total receptor concentration, and [PL] is the concentration of the receptor-bound ligand.

Under certain conditions, the receptor P can be characterized by a physical property denoted {P} with three characteristics: {P} has to be (a) macroscopically measurable, (b) linear under addition and multiplication by a constant, and (c) dependent only on the concentration of ligand-bound receptor and not on the concentration of the free ligand. Fluorescence observables such as amplitude averaged fluorescence lifetime, steady-state fluorescence intensity, and fluorescence anisotropy rotational lifetime satisfy these conditions. Hence, their physical property value {P}, at any initial ligand concentration is given by:

{P}([L₀])=(1−w([L₀]))*{P₀}+w([L₀])*{P_{∞}}={P₀}+w([L₀])*({P_{∞}}−{P₀}) (2)

where {P₀} is the value of the physical property in the absence of ligand, {P_{∞}} is the value of the physical property at saturating ligand concentration and, w([L₀]) is the fraction of ligand-bound receptor given by Eq. (1). Plugging Eq. (1) into Eq. (2) gives:

$$\{ P \} ( [ {L}_{0} ] ) = \{ {P}_{0} \} + \frac{\{ {P}_{∞} \} - \{ {P}_{0} \}}{1 + {( \frac{{K}_{D}}{[ {L}_{0} ]} )}^{n}} \qquad ( 3 )$$

For fluorescence lifetime measurements of metal binding to O-328 (18), O-328 (18) acts as the receptor and its concentration is kept constant to an indicated limiting value. Eq. (3) can be rewritten with {P}=t. For simplicity, the total concentration of ligand (i.e., metal ion) [L₀] is denoted as c. With these notations, Eq. (3) becomes:

$$τ ( c ) = {τ}_{0} + \frac{{τ}_{∞} - {τ}_{0}}{1 + {( \frac{{K}_{D}}{c} )}^{n}} \qquad ( 4 )$$

Since the metal ion concentration can span over many orders of magnitude (e.g., 1 nM-1 M), it is convenient to represent any concentration dependence by semi-log plots, with the log scale on the concentration axis and linear scale on the fluorescence lifetime axis. All the metal ion concentrations are normalized to (divided by) 1 nM and then the base 10 logarithm is taken. Log₁₀(c) is simply be denoted log(c). With these last considerations, the final equation for fitting the fluorescence lifetime data is derived as:

$$τ ( log ( c ) ) = {τ}_{0} + \frac{{τ}_{∞} - {τ}_{0}}{1 + {( \frac{log ( {K}_{D} )}{log ( c )} )}^{{n}^{*}}} \qquad ( 5 )$$

where n* is an apparent Hill coefficient once the x-axis is log-transformed. This value depends on the true Hill coefficient n, on the base that is chosen for the x-axis log-transform and on the value to which the x-axis is normalized. It is known that even in the case of a simple Michaelis-Menten hyperbola (n=1), once a semi-log plot is adopted the hyperbola becomes a sigmoidal curve. The K_{D} is also replaced by log(K_{D}) in order to maintain the meaning of this value, namely the ligand concentration at which half of the response between τ₀ and τ_{∞} is achieved.

Similarly, for fluorescence signal intensity measurements of metal binding to O-328 (18), the final formula for fitting of the fluorescence signal intensity data is derived as:

$$A ( log ( c ) ) = {A}_{0} + \frac{{A}_{∞} - {A}_{0}}{1 + {( \frac{log ( {K}_{D} )}{log ( c )} )}^{{n}^{*}}} \qquad ( 6 )$$

where n* is an apparent Hill coefficient once the x-axis is log-transformed. A₀ and A_{∞} define the dynamic range of the fluorescence signal change: the former represents the fluorescence signal in the absence of the potassium ion; the latter represents the fluorescence signal in the presence of saturating amount of the potassium ion. K_{D} is the dissociation constant of the potassium ion with O-328 (18). log(K_{D}) represent the KCl concentration at which half of the response between A₀ and A_{∞} is achieved.

### Example 5. Fluorescence Lifetime Measurements of O-328 (18) in the Presence of the Potassium Ion

Materials and Methods

Time-resolved fluorescence lifetime measurements of O-328 (18) containing an internal iCy3 (sequence: GAGGGACGG/iCy3/GGCAGGAGG) (SEQ ID NO:108) were performed as described in Example 2. The final concentration of O-328 (18) was 50 nM.

The fluorescence lifetimes of O-328 (18) in the presence of various concentrations of KCl (0-1 M) were plotted against the dimensionless log₁₀ of the respective KCl concentrations normalized to 1 nM. The resulting plot was fit to the Hill 1 function as described in Eq. 5 while fixing the initial fluorescence lifetime to that of the O-328 (18) sample in water. The reported dissociation constant (K_{D}) is the actual dissociation constant of the potassium ion from O-328 (18). n* is the apparent Hill coefficient determined from the fitting.

Time-resolved fluorescence lifetime measurements were also performed on another DNA oligonucleotide, which has the same nucleotide sequence as that of O-328 (18) but contains an internal iCy5 instead of an internal iCy3, i.e., GAGGGACGG/iCy5/GGCAGGAGG (SEQ ID NO:109). The samples were excited at 632 nm and emission was collected at 650 nm with 5 nm slit width for both excitation and emission. The final concentration of iCy5-labeled O-328 (18) was 50 nM.

Results

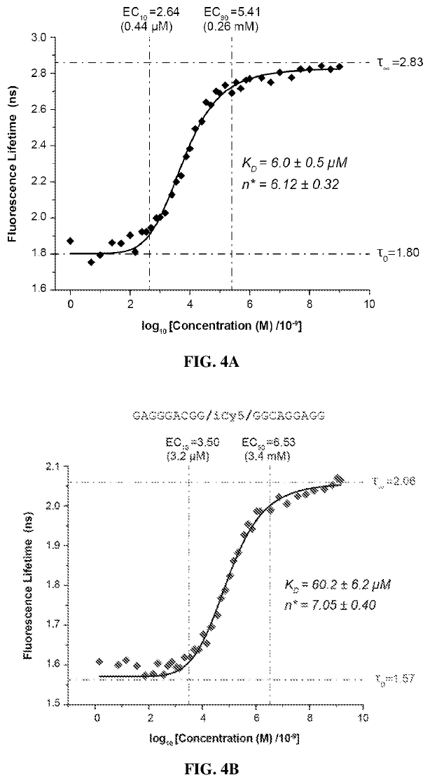

O-328 (18) exhibited a fluorescence lifetime of 1.8 ns in ultra-pure water in the absence of metal ions. The fluorescence lifetime continuously increased as the KCl concentration increased (FIG. 4A). This increase in iCy3's fluorescence lifetime reveals a conformational change within O-328 (18) that is K⁺-dependent and acts locally in a way that rigidifies iCy3, thus decreasing its photoisomerization rate and increasing the experimentally measured fluorescence lifetime.

The fluorescence lifetime values of O-328 (18) were plotted against the dimensionless log₁₀ of K⁺ concentration (FIG. 4A). The plot was fit using Eq. (5) with a fixed to value of 1.8 ns (the fluorescence lifetime in the absence of KCl). The fitting yielded a maximum fluorescence lifetime of 2.83 ns (the fluorescence lifetime at saturating KCl concentration). These two parameters gave the metal sensor a dynamic range of ˜1 ns. For the binding kinetics, the fitting yielded a microscopic dissociation constant K_{D} of ˜6 μM (3.77 in log₁₀ scale), suggesting that O-328 (18) is ultra-sensitive to the potassium ion in the low micro-molar range. The apparent Hill coefficient (n*) was determined to be ˜6, which represents the log₁₀ transformation of the concentration axis, not the true Hill coefficient.

The classical EC₁₀ and EC₉₀ values defined for sigmoidal response curves (Altszyler, et al. (2017) PLOS One, 12, e0180083) were also calculated for O-328 (18). The sigmoidal response curve in FIG. 4A showed an EC₁₀ value of 450 nM (2.64 in log₁₀ scale) and an EC₉₀ value of 260 μM (5.41 in log₁₀ scale), defining a working range that spans over 600 folds. This working range is transduced into 1 ns dynamic range of fluorescence lifetime.

Notably, the sensor design can be easily transferred to other environmental dyes. For example, FIG. 4B shows the result of the fluorescence lifetime measurement of an iCy5-containing DNA oligonucleotide having the same nucleotide sequence as that of O-328 (18), denoted as iCy5-O-328 (18). The fluorescence life-time response of iCy5-O-328 (18) is similar to the response of iCy3-labeled O-328 (18) (FIG. 1b). However, in the case of the former, the dynamic range of the change in fluorescence (˜31%) is smaller than that of the latter (˜57%). In addition, the K_{D} value for K⁺ is higher in iCy5-O-328 (18). The lower performance of the iCy5-O-328 (18) aptamer can be understood from the difference in the structures of Cy5 and Cy3. Cy5 has a longer polymethine moiety compared with Cy3, and is therefore more rigid. This rigidity makes Cy5 less environmentally sensitive than Cy3, which results in the decrease in the dynamic range of its change in fluorescence. The longer polymethine moiety may also influence the formation of a secondary structure of DNA, causing an increase in the K_{D} value for K⁺ binding.

### Example 6. Analysis of the Responsive Curve for O-328 (18)

Materials and Methods

For analysis of the responsive curve of O-328 (18), log(c) is treated as the independent x variable. This is possible since log(c) is a bijective function for any c>0.

The right hand side (R.H.S.) of Eq. (5) has useful mathematical properties. These properties were investigated between EC₁₀ and EC₉₀ since this range is considered as the working range for the sensor. The first and second derivatives of Eq. (5) are given respectively as the following:

$$\begin{matrix} \frac{\partial τ ( log ( c ) )}{\partial ( log ( c ) )} = \frac{{n}^{*}}{log ( c )} {( \frac{log ( {K}_{D} )}{log ( c )} )}^{{n}^{*}} \frac{{τ}_{∞} - {τ}_{0}}{{[ 1 + {( \frac{log ( {K}_{D} )}{log ( c )} )}^{{n}^{*}} ]}^{2}} & ( 7 ) \\ \frac{{\partial }^{2} τ ( log ( c ) )}{\partial {( log ( c ) )}^{2}} = \frac{{n}^{*}}{{( log ( c ) )}^{2}} {( \frac{log ( {K}_{d} )}{log ( c )} )}^{{n}^{*}} [ ( {n}^{*} - 1 ) {( \frac{log ( {K}_{D} )}{log ( c )} )}^{{n}^{*}} - {n}^{*} - 1 ] \frac{{τ}_{∞} - {τ}_{0}}{{[ 1 + {( \frac{log ( {K}_{D} )}{log ( c )} )}^{{n}^{*}} ]}^{3}} & ( 8 )\end{matrix}$$

Since all the factors of the R.H.S of Eq. (7) are positive, it follows that

$$\frac{\partial τ ( log ( c ) )}{\partial ( log ( c ) )} > 0 for c > 0.$$

The first derivative has a single maximum, given by the equation

$$\frac{{\partial }^{2} τ ( log ( c ) )}{\partial {( log ( c ) )}^{2}} = 0 ,$$

located at:

$$log ( {c}_{R} ) = log ( {K}_{D} ) {( \frac{{n}^{*} - 1}{{n}^{*} + 1} )}^{\frac{1}{{n}^{*}}} \qquad ( 9 )$$

For sigmoidal shaped curves, the local response of the curve was shown to be well described by a response coefficient function

$$R ( x ) = \frac{x}{y} \frac{dx}{dy}$$

(Altszyler, et al. (2017) PLOS One, 12, e0180083 and Kholodenko, et al. (1997) FEBS Letters, 414, 430-434). Considering the differential of the logarithm of base 10, i.e.,

$$d ( {log}_{10} ( x ) ) = \frac{dx}{x ln ( 10 )} \to \frac{x}{dx} = \frac{1}{ln ( 10 ) d ( {log}_{10} ( x ) )}$$

and replacing the variables, the response coefficient function becomes

$$R ( log ( c ) ) = \frac{1}{ln ( 10 ) τ ( log ( c ) )} \frac{\partial τ ( log ( c ) )}{\partial ( log ( c ) )} .$$

Introducing the derivative given by Eq. (7) into this response coefficient function yields:

$$R ( log ( c ) ) = \frac{1}{ln ( 10 ) [ {τ}_{0} + \frac{{τ}_{∞} - {τ}_{0}}{1 + {( \frac{log ( {K}_{D} )}{log ( c )} )}^{{n}^{*}}} ]} \frac{{n}^{*}}{log ( c )} {( \frac{log ( {K}_{D} )}{log ( c )} )}^{{n}^{*}} \frac{{τ}_{∞} - {τ}_{0}}{{[ 1 + {( \frac{log ( {K}_{D} )}{log ( c )} )}^{{n}^{*}} ]}^{2}} \qquad ( 10 )$$

A particular value of interest is:

$$R ( log ( c = {K}_{D} ) ) = \frac{{n}^{*} ( {τ}_{∞} - {τ}_{0} )}{2 ln ( 10 ) log ( {K}_{D} ) ( {τ}_{∞} + {τ}_{0} )} \qquad ( 11 )$$

Three simple observations can be made about this response value; the response (1) increases with the dynamic range of the measured physical property (R∝[(τ_{∞}−τ₀)/(τ_{∞}+τ₀)]), (2) increases with the Hill coefficient (R∝n*) and (3) decreases with the microscopic dissociation constant (R∝1/(log(K_{D})). These considerations show the important parameters for a binding-based sensor as a high dynamic range, high cooperativity and a low affinity constant. The maximum response value is comparable to that of the first derivative (given by Eq. (9)), which itself converges to log(K_{D}) for high enough n*, i.e.,

$${lim}_{{n}^{*} \to ∞} log ( {c}_{R} ) = log ( {K}_{D} ) .$$

One important characteristic of a response curve for a sensor is the reversibility of the response curve. Since the first derivative is positive everywhere and the function is continuous everywhere, it results that the R.H.S. of Eq. (5) is a bijective function and therefore is invertible. For any measured fluorescence lifetime, the inverse function is given by:

$$\{ log ( c ) \} ( τ ) = log ( {K}_{D} ) {( \frac{τ - {τ}_{0}}{{τ}_{∞} - {τ}_{0}} )}^{\frac{1}{{n}^{*}}} \qquad ( 12 )$$

The inverse function is also useful for calculating the relative error (uncertainty) in the concentration obtained by measuring a certain fluorescence lifetime and back-transducing it to concentration using Eq. (12). The first derivative of Eq. (12) is given by:

$$\frac{\partial \{ log ( c ) \} ( τ )}{\partial τ} = \frac{log ( {K}_{D} )}{{n}^{*}} \frac{{τ}_{∞} - {τ}_{0}}{( {τ}_{∞} - τ ) ( τ - {τ}_{0} )} {( \frac{τ - {τ}_{0}}{{τ}_{∞} - τ} )}^{\frac{1}{{n}^{*}}} \qquad ( 13 )$$

The uncertainty in {log(c)}(τ) can be approximated by Taylor series expansion as:

$$Δ \{ log ( c ) \} ( τ ) \approx \frac{\partial \{ log ( c ) \} ( τ )}{\partial τ} Δτ \qquad ( 14 )$$

where Δτ is the uncertainty of the time-resolved fluorescence measurements. Using the propagation of errors for the log function, i.e.,

$${Δlog}_{10} ( x ) \approx \frac{Δ x}{x ln ( 10 )} ,$$

we can write the relative error in the concentration as:

$$\frac{Δ c}{c} = ln ( 10 ) Δ \{ log ( c ) \} \qquad ( 15 )$$

Combining the last three equations gives this final result:

$$\{ \frac{Δ c}{c} ( τ ) \} ( \% ) = 100 ln ( 10 ) \frac{log ( {K}_{D} )}{{n}^{*}} \frac{{τ}_{∞} - {τ}_{0}}{( {τ}_{∞} - τ ) ( τ - {τ}_{0} )} {( \frac{τ - {τ}_{0}}{{τ}_{∞} - τ} )}^{\frac{1}{{n}^{*}}} Δτ \qquad ( 16 )$$

A particular value of interest is the error around the K_{D}, i.e., at τ=(τ_{∞}=+τ₀)/2. This value is then given by:

$$\{ \frac{Δ c}{c} ( \frac{{τ}_{∞} + {τ}_{0}}{2} ) \} ( \% ) = 100 ln ( 10 ) \frac{log ( {K}_{D} )}{{n}^{*}} \frac{4}{{τ}_{∞} - {τ}_{0}} Δτ \qquad ( 17 )$$

Four simple observations can be made about this response value; the error (1) decreases with the dynamic range of the measured physical property (Δc/c∝1/(τ_{∞}−τ₀)), (2) decreases with the Hill coefficient (Δc/c∝1/n*), (3) increases with the microscopic dissociation constant (Δc/c∝log(K_{D})) and (4) increases with the uncertainty of the measurement (Δc/c∝Δτ). Except for the dependency on the instrument's resolution, the relative error shows the opposite behavior of the response coefficient function.

Results

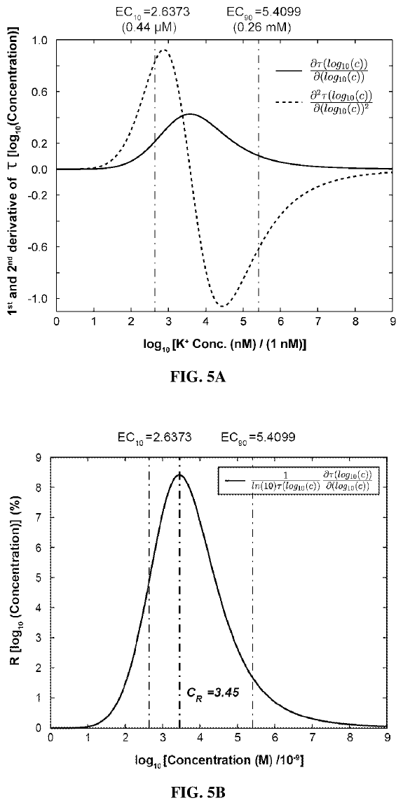

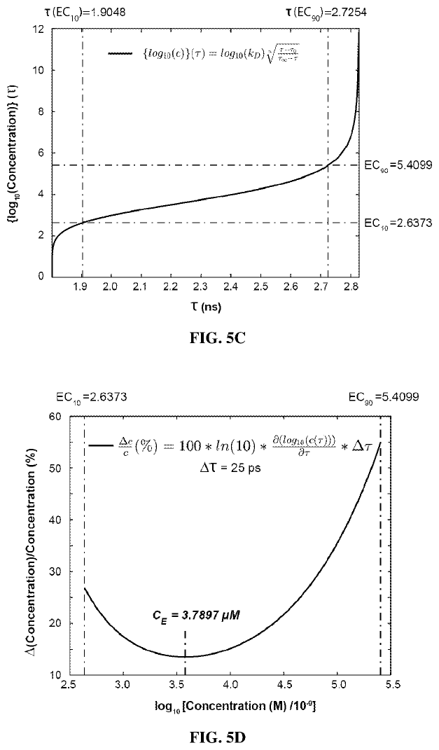

To further characterize the K⁺ sensor O-328 (18), the properties of its response curve in the interval between EC₁₀ and EC₉₀ were investigated. The first and second derivatives of the response curve were plotted according to Eqs. (7) and (8), as shown in FIG. 5A. The first derivative is positively defined everywhere, indicating that the response function is a strictly monotonically increasing function. Furthermore, the first derivative has a single global maximum, which can be solved for by setting the second derivative to zero as given by Eq. (9). Since n* is high, this global maximum approaches the value of K_{D}. This alludes that the maximum sensitivity of the sensor is around K_{D}.

If the response curve is investigated on a short interval around the K_{D}, the first two derivatives would give a good enough approximation of the response by Taylor series expansion. To investigate the local response over the whole concentration dependence the formalism of a local response coefficient for sigmoidal curves was adopted as described earlier (Kholodenko, et al. (1997) FEBS Letters, 414, 430-434). The definition of the coefficient function was changed to reflect the sensor's lifetime dynamic range as shown in Eq. (10). The resulting curve (FIG. 5B) shows a single-peak function, similar to the first derivative, with the peak centered at ˜3 μM (3.45 in log₁₀ scale), relatively comparable to the K_{D} (6 μM). Between EC₁₀ and EC₉₀, the sensor offers 2-9% (FIG. 5B) local response to the variation of concentration (absolute concentration values not in log₁₀ scale). Considering the high temporal resolution of the current time-resolved detectors, this response is significant. This local response coefficient indicates that if the concentration is varied by a certain percentage, the response will vary by that percentage multiplied with the local response coefficient of the initial concentration of the variance. For example, the local response coefficient would predict that doubling the concentration from 3 μM to 6 μM would result in 9% change in the fluorescence lifetime. This 9% change translates to ˜200 ps, which is easily resolvable by a detector with 25 ps time resolution.

The response curve illustrated in FIG. 5B is very useful as a calibration curve. However, using a particular instrument and experimental conditions, it is recommended to use the inverse curve for estimating unknown samples. Mathematically, this is only possible if and only if the response curve is invertible, such that to any metal ion concentration corresponds one and only one fluorescence lifetime. The response curve is a strictly monotonically increasing function, as described earlier, as well as continuous everywhere. It follows then that the response function is a bijective function and therefore, invertible. Simply put, the inverse function, described by Eq. (12) and plotted in FIG. 5C, takes a measured fluorescence lifetime of the O-328 (18) sensor in an unknown K⁺-containing solution and outputs the corresponding K⁺ concentration. This curve exhibited almost linear relationship between log₁₀ of concentration (y-axis) and fluorescence lifetime (x-axis) in the interval between EC₁₀ and EC₉₀. Furthermore, this curve defined the dynamic range of fluorescence lifetime (1.9-2.7 ns) required for the 0328 (18) sensor to operate in the interval between EC₁₀ and EC₉₀.

Further, a theoretical estimation of the uncertainty associated with measuring an unknown K⁺ concentration using the O-328 (18) sensor was determined. The relative uncertainty is given by Eq. (17) and plotted in FIG. 5D. This relative uncertainty is dictated by the parameters of the response curve as well as the uncertainty of the lifetime measurement. The relative error varies between 15% and 55% in the interval between EC₁₀ and EC₉₀, assuming an uncertainty of the lifetime of 25 ps dictated by the instrument detector. The curve exhibits a single local minimum located at around 3.8 μM (3.58 in log₁₀ scale) close to the value of K_{D} (6 μM).

Overall, the properties of the response curve (FIGS. 5A-5D) suggest that the O-328 (18) sensor excels in both maximizing the response and minimizing the uncertainty around the K_{D} value of 6 μM. Nonetheless, the sensor is effective over the whole interval between EC₁₀ and EC₉₀. However, for a very precise measurement of an unknown sample, the sample can be serially diluted to a K⁺ concentration around K_{D} or equivalently to a measured lifetime of O-328 (18) around 2.3 ns.

### Example 7. Steady-State Fluorescence Measurement of O-328 (18) in the Presence of the Potassium Ion

Materials and Methods

Steady-state fluorescence measurements of O-328 (18) containing an internal i-Cy3 were performed at room temperature using a microplate spectrofluorometer (TECAN infinite M1000). The final concentration of O-328 (18) was 50 nM. The samples were excited at 535 nm (λₘₐₓ₋ₑₓ of iCy3). Full emission spectra were collected between 520 and 700 nm. Fixed-wavelength fluorescence intensity was only recorded at 565 nm (λₘₐₓ₋ₑₘ of iCy3) for plotting the K⁺-dependent response plot. Excitation and emission slit widths were set to 5 nm, and measurements were acquired with an integration time of 0.1 s. The emission spectra and intensities were corrected by subtracting the background emission of a water blank. The spectra were further smoothened using fast Fourier transform (FFT) implemented in Origin-pro software. The noise harmonics were determined at a dynamic window size of 5 nm corresponding to the slit width.

In FIG. 2A, the fluorescence signal change of O-328 (18) at different KCl concentrations were plotted against the dimensionless log₁₀ of KCl concentration normalized to 1 nM. This plot was fitted to a Hill 1 function as shown in Eq. (6). Error bars correspond to the variation in the measurement between two replicates.

The absorbance of O-328 (18) in increasing concentrations of KCl was measured at room temperature using a microplate spectrophotometer (TECAN infinite M1000). The concentration of O-328 (18) was kept at 1 μM such that the absorbance was below 0.1 to minimize the reabsorption effect. The absorption spectra were acquired from 440 to 600 nm. Criteria of 1 nm wavelength step size along with 100 flashes per step were used. These spectra were corrected by measuring the instrumental baseline with a water blank. Similar to the fluorescence emission spectra, the absorption spectra were smoothened using FFT. Absorbance was quantified by integrating over the entire spectra. Absorption change due to increasing salt concentration was calculated as a percentage with respect to the absorbance of the sample containing no salt.

Results

Steady-state fluorescence spectra (520-700 nm) of O-328 (18) at various K⁺ concentrations were collected. The spectra did not exhibit spectral-chromatic changes as the K⁺ concentration increased to 1 M.

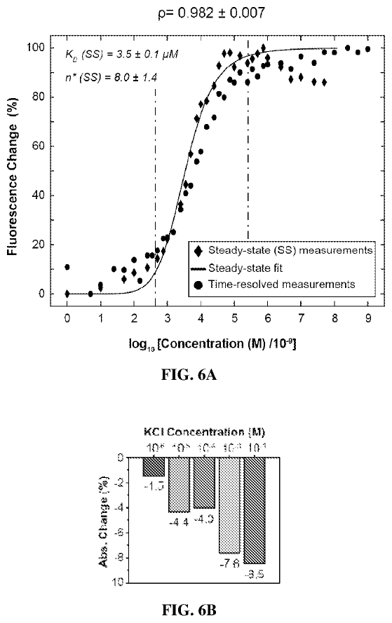

Analogous to the lifetime measurements, the steady-state fluorescence intensities increased continuously as the K⁺ concentration increased from 0 to 1 mM (FIG. 6A). Plotting and fitting this response curve to a Hill 1 function as shown in Eq. (6) yielded a K_{D-SS} value of ˜3.5 M, a value that is comparable to that generated by the fluorescence lifetime response curve. The steady-state response curve also showed an apparent Hill coefficient n*_{SS} of ˜8. The difference in the apparent Hill coefficient can be attributed to the faster saturation of the steady-state response curve. However, unlike the lifetime measurements, the steady-state fluorescence intensities slightly decreased as the K⁺ concentration increased beyond 1 mM.

For a better comparison between the time-resolved and steady-state measurements, the response curves of fluorescence lifetime and fluorescence signal intensity was normalized to values between 0 and 1 (0% and 100%) in order to keep only the binding dependence (Eq. (1)) and exclude the dynamic range of each measurement (FIG. 6A). It is worthy to note that the slight decrease of steady-state fluorescence at high K⁺ concentrations is irrelevant since these concentrations are beyond the EC₁₀-EC₉₀ interval. Thus, Pearson correlation coefficient between steady-state and time-resolved responses was calculated for data points that fall within EC₁₀-EC₉₀ interval in order to analytically assess the agreement between the two measurements. This Pearson correlation coefficient of ˜0.98 showed high agreement between the measurements, and therefore, the response curve can be reproduced by either approach.

To better understand the steady-state behavior, the absorption spectra of O-328 (18) under various K⁺ concentrations were collected. These spectra did not exhibit any spectral-chromatic changes at varying K⁺ concentrations and their integrated absorbance showed a slight decrease up to 8.5% at 100 mM K⁺ (FIG. 6B). For K⁺ concentrations below 1 mM, the fluorescence signal intensity increased as the K⁺ concentration increased; thus, the observed absorbance decrease in this K⁺ concentration range had no effect on the fluorescence signal. However, as the K⁺ concentration increased beyond 1 mM, the absorbance decrease can explain the decrease in the steady-state fluorescence.

In conclusion, this set of experiments suggests that the O-328 (18) sensor can function under both the time-resolved and steady-state approaches, particularly in the EC₁₀-EC₉₀ interval. Furthermore, the decrease in steady-state fluorescence emission at K⁺ concentrations higher than 1 mM cannot be explained by a collisional quenching mechanism, since the measured fluorescence lifetime is not affected. Further explanation of fluorescence quenching mechanisms can be found in Fraiji, et al. (1992) Journal of Chemical Education, 69, 424. It is anticipated that this decrease in steady-state fluorescence could stem from a combination of static quenching, reflected by the absorbance decrease, and possible alterations to the oligonucleotide structure triggered by the high K⁺ concentrations.

### Example 8. Fluorescence Lifetime Measurements of O-328 (18) in the Presence of Other Metal Ions Materials and Methods

The fluorescence lifetime of O-328 (18) was also measured in the presence of various metal ions (10 mM), including lithium, magnesium, manganese, calcium, cesium, zinc, sodium, potassium, and strontium. A non-metal cation, ammonium, was also tested. The final concentration of O-328 (18) was 50 nM. Data were collected as described in Example 2.

Concentration-dependent fluorescence lifetime measurements of O-328 (18) were also performed for the ammonium cation and the sodium ion. The experimental data were fit to Eq. 5 as described in Example 5.

Results

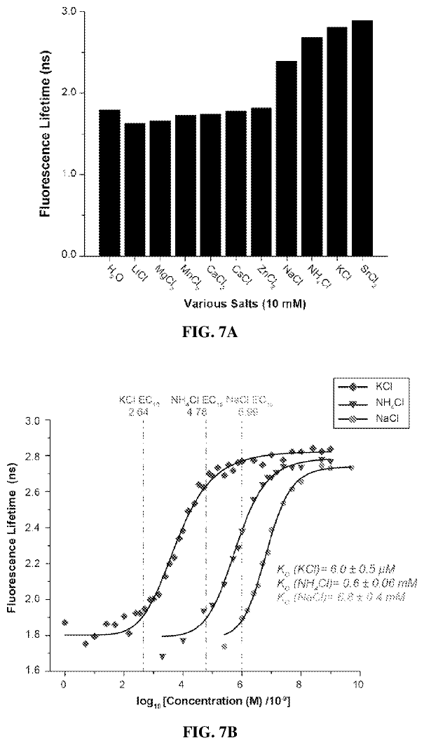

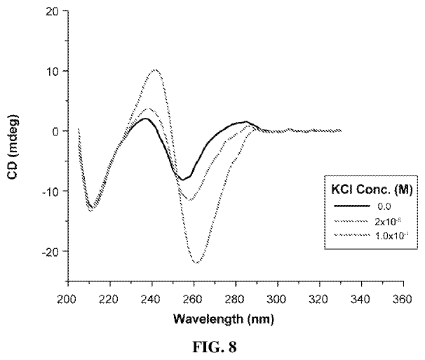

DNA secondary structures, especially G-quadruplexes, have been shown to coordinate multiple mono- and divalent metal ions (Bhattacharyya, et al. (2016) Frontiers in Chemistry, 4, 38). O-328 (18)'s versatility in coordinating different metal ions other than K⁺ was investigated. The fluorescence lifetime of O-328 (18) was measured in the presence of 10 mM of various cations (Li⁺, Mg²⁺, Mn²⁺, Ca²⁺, Cs⁺, Zn²⁺, Na⁺, NH₄⁺, K⁺ and Sr²⁺) in the form of chloride salts (FIG. 7A). Chloride anion was maintained for all the tested salts to exclusively assess the effect of the cations. In the presence of Li⁺, Mg²⁺, Mn²⁺, Ca²⁺, Cs⁺, and Zn²⁺, the fluorescence lifetime of O-328 (18) did not change or slightly decreased indicating that these cations did not stabilize a DNA secondary structure as was suggested earlier for G-quadruplexes (Bhattacharyya, et al. (2016) Frontiers in Chemistry, 4, 38; Blume, et al. (1997) Nucleic Acids Research, 25, 617-625; Hardin, et al. (2000) Biopolymers, 56, 147-194; and Neidle, et al. (2006) Quadruplex Nucleic Acids, Cambridge: RSC: Biomoleculer Sciences). On the other hand, the fluorescence lifetime of O-328 (18) increased in the presence of Na⁺, NH₄⁺, K⁺, and Sr²⁺ suggesting that these cations induced and stabilized a DNA secondary structure. It is worth mentioning that in the context of the O-328 (18) sensor, Sr²⁺ pushes the fluorescence lifetime of iCy3 close to the theoretical lifetime of Cy3B (3.19 ns) in the absence of any non-radiative loss (Cooper, et al. (2004) Journal of Fluorescence, 14, 145-150).

Taking into consideration that Na⁺ and NH₄⁺ are commonly encountered cations, fluorescence lifetimes of the O-328 (18) sensor in increasing concentrations of these two cations were measured. The response curves for these two cations exhibited similar sigmoidal behavior to that of K⁺ and were fitted using Eq. (5) (FIG. 7B). Based on the generated K_{D} values, the O-328 (18) sensor is 100 fold less sensitive to NH₄⁺ (K_{D}=0.6 mM) and 1000 fold less sensitive to Na⁺ (K_{D}=6.8 mM), compared to K⁺.

### Example 9. Circular Dichroism Measurements of O-328 (18)

Materials and Methods

The circular dichroism (CD) spectra of unlabeled O-328 (18) in which the iCy3 dye was replaced by a thymidine deoxynucleotide (sequence: GAGGGACGG/T/GGCAGGAGG (SEQ ID NO:110)) in increasing concentrations of KCl was measured at room temperature using a CD spectrophotometer (JASCO J-1500). The unlabeled O-328 concentration was kept at 20 μM to ensure a reliable signal-to-noise ratio. The CD spectra were acquired from 205 to 350 nm. Criteria of 1-nm wavelength step size along with 50 nm/min scanning speed were used. These spectra were corrected by subtracting the instrumental baseline obtained with a water blank. Similar to the fluorescence emission spectra, the CD spectra were smoothened using FFT. The total UV absorption was also simultaneously monitored with the CD measurement to monitor the consistency of the oligo concentration.

Results

FIG. 8 shows the CD spectra (220-350 nm) of unlabeled O-328 (18) in the presence of different concentrations of KCl. The inset of the figure shows that the absorbance of unlabeled O-328 (18) did not change upon addition of the potassium ion. However, the CD spectrum of unlabeled O-328 (18) was significantly altered by the addition of the potassium ion. It is evident that a specific secondary structure was induced by the potassium ion in unlabeled O-328 (18) in a concentration-dependent manner. This result suggests that the potassium-induced structural change in O-328 is not an artifact due to the presence of the iCy3 dye.

### Example 10. Single Molecule Fluorescence Measurements of O-328 (22)

Materials and Methods

Single molecule measurements followed similar protocols as described in Rashid, et al. (2017) eLife, 6, e21884; Zaher, et al. (2018) Nucleic Acids Research, 46, 2956-2974; and Sobhy, et al. (2013) Cell Reports, 3, 1785-1794. Briefly, the measurements were all performed at room temperature in a custom airtight microfluidic flow cell with a glass coverslip that was functionalized and passivated by 1:100 molar ratio of biotinylated polyethylene glycol (Biotin-PEG-SVA MW 5,000) and polyethylene glycol (mPEG-SVA MW 5000) (Laysan Bio Inc.). DNA substrates (100-200 pM) were immobilized onto the surface using biotin-NeutrAvidin interaction. Prior to the DNA immobilization, the surface was incubated with 0.2 mg/ml NeutrAvidin for 10-15 min followed by excessive washing with reaction buffer to remove excess NeutrAvidin and block any extra unspecific binding sites. To enhance the fluorophores' photostability and reduce photo-blinking, the imaging buffer included a mixture of the reaction buffer (with or without the potassium ion), 2 mM Trolox (Sigma-Aldrich), and an oxygen scavenging solution as described in Aitken, et al. (2008) Biophysical Journal, 94, 1826-1835. All single molecule experiments were performed using a custom-built TIRF-FRET setup as described in Sobhy, et al. (2011) The Review of Scientific Instruments, 82, 113702. Several movies of each condition were recorded on different fields of view in two-color alternating excitation (2c-ALEX) mode and/or continuous excitation mode, as described in Kapanidis, et al. (2005) Accounts of Chemical Research, 38, 523-533. The time resolution for the different experiments is mentioned in their respective figure legends. Data extraction using twotone software (see Kapanidis, et al. (2005) Accounts of Chemical Research, 38, 523-533.) followed the protocols described previously in Rashid, et al. (2017) eLife, 6, e21884 and Zaher, et al. (2018) Nucleic Acids Research, 46, 2956-2974.

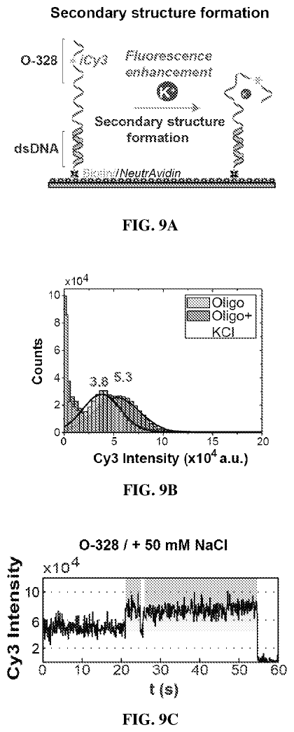

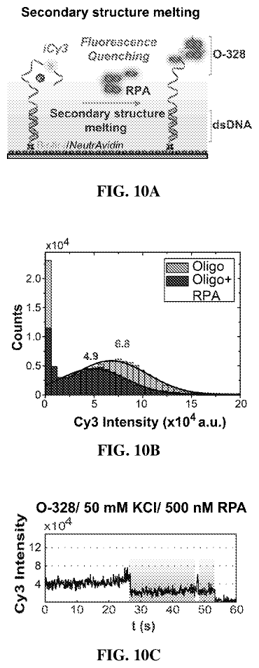

As illustrated in FIG. 9A, the DNA substrate used was a primer-template (P/T) junction composed of a long (nt) iCy3-labeled oligonucleotide containing the O-328 (22) sequence (i.e., AGGAGGGACGG/iCy3/GGCAGGAGGAG (SEQ ID NO:107)) at its 5′end and annealed to a biotinylated complimentary short (nt) oligo at its 3′end. This substrate was immobilized to the surface through biotin-NeutrAvidin interaction with the dsDNA region near the surface and the ssDNA region containing O-328 (22) extending further away from the surface.

For monitoring the secondary structure formation, the DNA substrate (100-200 pM) was first immobilized to the surface in a reaction buffer excluding KCl (50 mM HEPES, pH=7.5, 5% glycerol, 1 mM MgCl₂). Three movies of different fields of view were recorded at equilibrium using continuous excitation of green laser. Second, a reaction buffer containing 50 mM KCl (50 mM HEPES, pH=7.5, 5% glycerol, 50 mM KCl, 1 mM MgCl₂) was injected into the flow cell. Prior to the arrival of the KCl-containing buffer to the flow cell, recording was started under continuous flow of buffer. Finally, three movies of different fields of view were recorded after equilibrium with the exchanged buffer was reached. The movies taken at equilibrium before and after the injection of the reaction buffer containing 50 mM KCl were used to construct the distributions of iCy3 fluorescence intensity in the two conditions. These distributions were fit with Gaussian peaks using OriginPro and the center of these peaks were reported (FIG. 9B). The movie recorded under continuous flow was used to monitor the change of iCy3 fluorescence, in real time, as shown in the time trace of FIG. 9C.

Similarly, to observe the melting of the K⁺-induced secondary structure of O-328 (22), the same P/T substrate was immobilized to the surface in a reaction buffer containing 50 mM KCl. Three movies were recorded, at equilibrium, before the injection of 100 nM human replication protein A (RPA) in the presence of 50 mM KCl. Human RPA is an ssDNA-binding protein that can bind to O-328 (22). A movie was recorded, starting prior to RPA arrival to the flow cell and under continuous flow. At last, three movies were recorded after the final equilibrium with RPA was reached. These movies were used to construct the iCy3 intensity histograms and time traces, in a similar fashion to those described for the formation of the secondary structure.

Results

A longer oligonucleotide containing the sequence of O-328 (22) at one end is annealed to a biotinylated oligo at the other end, creating a P/T junction (FIG. 9A). This ensured that the fluorescent end of O-328 (22) was free and away from the surface. This design provided a well-spaced surface, where the formation of a dimer was almost impossible. In the absence of K⁺, the P/T junction showed a low fluorescence intensity profile, centered around 3.8×10⁴ a.u. (FIG. 9B). The addition of 50 mM KCl clearly shifted the fluorescence intensity distribution to a higher fluorescent regime, centered around 5.3×10⁴ a.u. (40% increase), similarly to what we observed using bulk measurements. More importantly, the time trace upon the injection of the potassium-containing reaction buffer showed an instantaneous transition to a higher fluorescence state (FIG. 9C). This indicates that the K⁺-induced fluorescence enhancement is a single-step process (within the temporal resolution) rather than a progressive process that goes through multiple states to reach the maximum fluorescence state. Moreover, the time trace in FIG. 9C exhibited one-step photo-bleaching, further confirming the monomer nature of the P/T junction.

FIG. 10B shows that addition of human RPA in the presence of KCl quenched the fluorescence intensity of iCy3 from 6.8×10⁴ a.u. to 4.9×10⁴ a.u. (a 28% disease), indicating that RPA is capable of melting the secondary structure of O-328 (22) and reversing the K⁺-induced fluorescence enhancement to a lower intensity profile as illustrated in FIG. 10A. The time trace showed that both the RPA-induced fluorescence quenching and the photo-bleaching event afterwards were single-step processes (FIG. 10C).

Taken together, it is concluded that the K⁺-induced fluorescence enhancement of O-328 (22) is caused by a change in the overall structure of this iCy3-labeled oligonucleotide, through the formation of a specific secondary structure similar to G-quadruplexes as described in Campbell, et al. (2012) Metal Ions in Life Sciences, 10, 119-134. This secondary structure can be perturbed by human RPA, which can bind to the DNA oligonucleotide and disrupt the K⁺-induced secondary structure.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A single-stranded oligonucleotide, comprising one of the following sequences:
SEQ ID NO. 1
AGGAGGGACGGGGCAGGAGGAG
SEQ ID NO. 2
GGAGGGACGGGGCAGGAGGAG
SEQ ID NO. 3
AGGAGGGACGGGGCAGGAGGA
SEQ ID NO. 4
GAGGGACGGGGCAGGAGGAG
SEQ ID NO. 5
GGAGGGACGGGGCAGGAGGA
SEQ ID NO. 6
AGGAGGGACGGGGCAGGAGG
SEQ ID NO. 7
AGGGACGGGGCAGGAGGAG
SEQ ID NO. 8
GAGGGACGGGGCAGGAGGA
SEQ ID NO. 9
GGAGGGACGGGGCAGGAGG
SEQ ID NO. 10
AGGAGGGACGGGGCAGGAG
SEQ ID NO. 11
GGGACGGGGCAGGAGGAG
SEQ ID NO. 12
AGGGACGGGGCAGGAGGA
SEQ ID NO. 13
GAGGGACGGGGCAGGAGG
SEQ ID NO. 14
GGAGGGACGGGGCAGGAG
SEQ ID NO. 15
AGGAGGGACGGGGCAGGA
SEQ ID NO. 16
GGGACGGGGCAGGAGGA
SEQ ID NO. 17
AGGGACGGGGCAGGAGG
SEQ ID NO. 18
GAGGGACGGGGCAGGAG
SEQ ID NO. 19
GGAGGGACGGGGCAGGA
SEQ ID NO. 20
GGGACGGGGCAGGAGG
SEQ ID NO. 21
AGGGACGGGGCAGGAG
SEQ ID NO. 22
GAGGGACGGGGCAGGA
SEQ ID NO. 23
GGGACGGGGCAGGAG
SEQ ID NO. 24
AGGGACGGGGCAGGA
SEQ ID NO. 25
GGGACGGGGCAGGA

2. The oligonucleotide of claim 1, having between 14 and 22, inclusive, nucleotides, and/or wherein the oligonucleotide is a DNA oligonucleotide.

3. (canceled)

4. A reporter-containing oligonucleotide, comprising
(a) a nucleotide sequence in which at least 40, 45, 50, 55, 60, 65, or 70 percent of the nucleotides are guanine nucleotides; and
(b) a fluorescence dye as a built-in reporter,
wherein the fluorescence dye is incorporated in the sugar-phosphate backbone of the oligonucleotide,
wherein the oligonucleotide optionally undergoes a conformational change upon binding to a ligand of the oligonucleotide, wherein the conformational change of the oligonucleotide induces a photophysical change of the fluorescence dye.

5. The reporter-containing oligonucleotide of claim 4, wherein the oligonucleotide forms a G-quadrulex upon binding the ligand.

6. The reporter-containing oligonucleotide of claim 4, comprising one of the following sequences:
SEQ ID NO. 26
AGGAGGGACGG/X/GGCAGGAGGAG
SEQ ID NO. 27
GGAGGGACGG/X/GGCAGGAGGAG
SEQ ID NO. 28
AGGAGGGACGG/X/GGCAGGAGGA
SEQ ID NO. 29
GAGGGACGG/X/GGCAGGAGGAG
SEQ ID NO. 30
GGAGGGACGG/X/GGCAGGAGGA
SEQ ID NO. 31
AGGAGGGACGG/X/GGCAGGAGG
SEQ ID NO. 32
AGGGACGG/X/GGCAGGAGGAG
SEQ ID NO. 33
GAGGGACGG/X/GGCAGGAGGA
SEQ ID NO. 34
GGAGGGACGG/X/GGCAGGAGG
SEQ ID NO. 35
AGGAGGGACGG/X/GGCAGGAG
SEQ ID NO. 36
GGGACGG/X/GGCAGGAGGAG
SEQ ID NO. 37
AGGGACGG/X/GGCAGGAGGA
SEQ ID NO. 38
GAGGGACGG/X/GGCAGGAGG
SEQ ID NO. 39
GGAGGGACGG/X/GGCAGGAG
SEQ ID NO. 40
AGGAGGGACGG/X/GGCAGGA
SEQ ID NO. 41
GGGACGG/X/GGCAGGAGGA
SEQ ID NO. 42
AGGGACGG/X/GGCAGGAGG
SEQ ID NO. 43
GAGGGACGG/X/GGCAGGAG
SEQ ID NO. 44
GGAGGGACGG/X/GGCAGGA
SEQ ID NO. 45
GGGACGG/X/GGCAGGAGG
SEQ ID NO. 46
AGGGACGG/X/GGCAGGAG
SEQ ID NO. 47
GAGGGACGG/X/GGCAGGA
SEQ ID NO. 48
GGGACGG/X/GGCAGGAG
SEQ ID NO. 49
AGGGACGG/X/GGCAGGA
SEQ ID NO. 50
GGGACGG/X/GGCAGGA
wherein X represents the fluorescence dye.

7. The reporter-containing oligonucleotide of claim 4, having between 14 and 22, inclusive, nucleotides and/or wherein the oligonucleotide is a DNA oligonucleotide.

8. (canceled)

9. The reporter-containing oligonucleotide of claim 4, wherein the fluorescence dye is an environmentally sensitive fluorescence dye.

10. The reporter-containing oligonucleotide of claim 9, wherein the environmentally sensitive fluorescence dye is a cyanine dye.

11. The reporter-containing oligonucleotide of claim 10, wherein the cyanine dye has a structure selected from the group selected from the group consisting of the following formula or a derivative thereof: 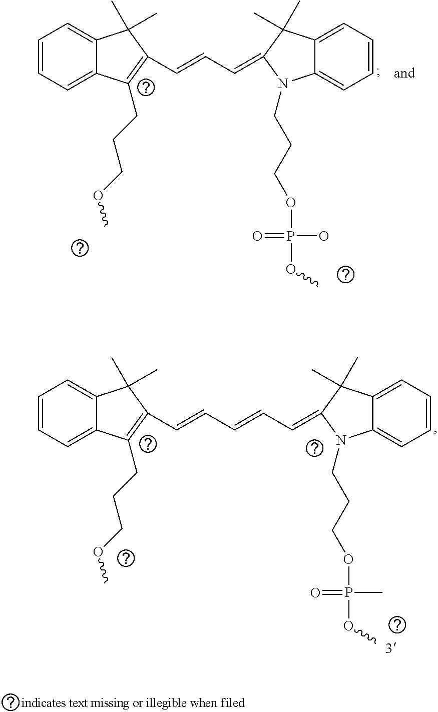
or a derivative thereof

12. (canceled)

13. The reporter-containing oligonucleotide of claim 4, wherein the ligand is a metal ion, optionally selected from the group consisting of potassium, lithium, magnesium, manganese, calcium, cesium, zinc, sodium, potassium, and strontium.

14. The reporter-containing oligonucleotide of claim 4, wherein the photophysical change of the fluorescence dye is a shift in the fluorescence wavelength, a change in the fluorescence signal intensity, a change in the fluorescence life time, or combinations thereof.

15. The reporter-containing oligonucleotide of claim 14, wherein the fluorescence dye is non-fluorescent or fluorescent-quenched prior to binding of the ligand and becomes fluorescent or fluorescent-enhanced upon binding of the ligand.

16. A method to detect a target molecule, comprising:
(a) contact a sample containing the target molecule with a single-stranded reporter-containing oligonucleotide comprising a sequence in which at least 40, 45, 50, 55, 60, 65, or 70 percent of the nucleotides are guanine nucleotides and a fluorescence dye as a built-in reporter, wherein the fluorescence dye is incorporated in the sugar-phosphate backbone of the sequence of the oligonucleotide via covalent linkages with its neighboring nucleotides;
(b) detect photophysical change of the fluorescence dye,
wherein detection of the photophysical change of the fluorescence dye indicates conformational change of the oligonucleotide induced by binding to the target molecule.

17. The method of claim 16, wherein the oligonucleotide comprises a sequence selected from SEQ ID NO. 26-50.

18. The method of claim 16, wherein: (a) the oligonucleotide has between 14 and 22, inclusive, nucleotides: (b) the oligonucleotide is a DNA oligonucleotide; (c) the fluorescence dye is an environmentally sensitive fluorescence dye.

19. (canceled)

20. (canceled)

21. The method of claim 18, wherein the environmentally sensitive fluorescence dye is a cyanine dye.

22. The method of claim 21, wherein the cyanine dye has a structure selected from the group consisting of 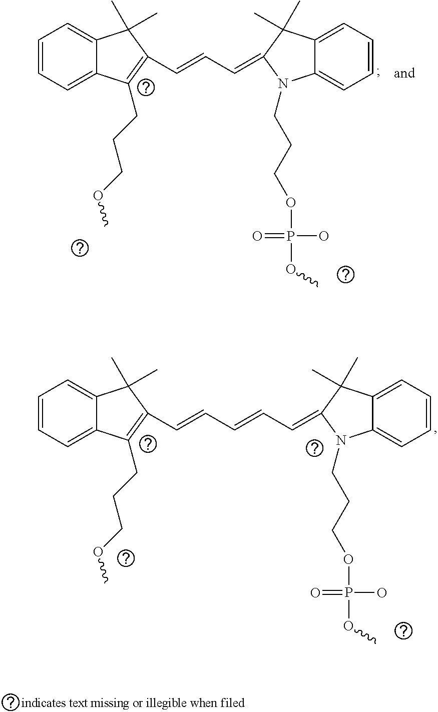
or a derivative

23. (canceled)

24. The method of claim 16, wherein: (a) the target molecule is a metal ion, optionally selected from the group consisting of potassium, lithium, magnesium, manganese, calcium, cesium, zinc, sodium, potassium, and strontium; and/or (b) the photophysical change of the fluorescence dye is a shift in the fluorescence wavelength, a change in the fluorescence signal intensity, a change in the fluorescence life time, or combinations thereof.

25. The method of claim 24, wherein the metal ion is potassium.

26. (canceled)

27. The method of claim 26, wherein the fluorescence dye is non-fluorescent or fluorescent-quenched prior to binding of the target molecule and becomes fluorescent or fluorescent-enhanced upon binding of the target molecule.


SEQUENCE LISTING (110 sequences)

SEQ ID NO: 1 - DNA, 22 bp, Artificial Sequence
  Synthetic nucleotide
aggagggacggggcaggaggag

SEQ ID NO: 2 - DNA, 21 bp, Artificial Sequence
  Synthetic nucleotide
ggagggacggggcaggaggag

SEQ ID NO: 3 - DNA, 21 bp, Artificial Sequence
  Synthetic nucleotide
aggagggacggggcaggagga

SEQ ID NO: 4 - DNA, 20 bp, Artificial Sequence
  Synthetic nucleotide
gagggacggggcaggaggag

SEQ ID NO: 5 - DNA, 20 bp, Artificial Sequence
  Synthetic nucleotide
ggagggacggggcaggagga

SEQ ID NO: 6 - DNA, 20 bp, Artificial Sequence
  Synthetic nucleotide
aggagggacggggcaggagg

SEQ ID NO: 7 - DNA, 19 bp, Artificial Sequence
  Synthetic nucleotide
agggacggggcaggaggag

SEQ ID NO: 8 - DNA, 19 bp, Artificial Sequence
  Synthetic nucleotide
gagggacggggcaggagga

SEQ ID NO: 9 - DNA, 19 bp, Artificial Sequence
  Synthetic nucleotide
ggagggacggggcaggagg

SEQ ID NO: 10 - DNA, 19 bp, Artificial Sequence
  Synthetic nucleotide
aggagggacggggcaggag

SEQ ID NO: 11 - DNA, 18 bp, Artificial Sequence
  Synthetic nucleotide
gggacggggcaggaggag

SEQ ID NO: 12 - DNA, 18 bp, Artificial Sequence
  Synthetic nucleotide
agggacggggcaggagga

SEQ ID NO: 13 - DNA, 18 bp, Artificial Sequence
  Synthetic nucleotide
gagggacggggcaggagg

SEQ ID NO: 14 - DNA, 18 bp, Artificial Sequence
  Synthetic nucleotide
ggagggacggggcaggag

SEQ ID NO: 15 - DNA, 18 bp, Artificial Sequence
  Synthetic nucleotide
aggagggacggggcagga

SEQ ID NO: 16 - DNA, 17 bp, Artificial Sequence
  Synthetic nucleotide
gggacggggcaggagga

SEQ ID NO: 17 - DNA, 17 bp, Artificial Sequence
  Synthetic nucleotide
agggacggggcaggagg

SEQ ID NO: 18 - DNA, 17 bp, Artificial Sequence
  Synthetic nucleotide
gagggacggggcaggag

SEQ ID NO: 19 - DNA, 17 bp, Artificial Sequence
  Synthetic nucleotide
ggagggacggggcagga

SEQ ID NO: 20 - DNA, 16 bp, Artificial Sequence
  Synthetic nucleotide
gggacggggcaggagg

SEQ ID NO: 21 - DNA, 16 bp, Artificial Sequence
  Synthetic nucleotide
agggacggggcaggag

SEQ ID NO: 22 - DNA, 16 bp, Artificial Sequence
  Synthetic nucleotide
gagggacggggcagga

SEQ ID NO: 23 - DNA, 15 bp, Artificial Sequence
  Synthetic nucleotide
gggacggggcaggag

SEQ ID NO: 24 - DNA, 15 bp, Artificial Sequence
  Synthetic nucleotide
agggacggggcagga

SEQ ID NO: 25 - DNA, 14 bp, Artificial Sequence
  Synthetic nucleotide
gggacggggcagga

SEQ ID NO: 26 - DNA, 22 bp, Artificial Sequence
  Synthetic nucleotide
  misc_feature (11)..(12): fluroescent dye
aggagggacggggcaggaggag

SEQ ID NO: 27 - DNA, 21 bp, Artificial Sequence
  Synthetic nucleotide
  misc_feature (10)..(11): Fluroescent dye
ggagggacggggcaggaggag

SEQ ID NO: 28 - DNA, 21 bp, Artificial Sequence
  Synthetic nucleotide
  misc_feature (11)..(12): Fuorescence dye
aggagggacggggcaggagga

SEQ ID NO: 29 - DNA, 20 bp, Artificial Sequence
  Synthetic nucleotide
  misc_feature (9)..(10): Fluorescent dye
gagggacggggcaggaggag

SEQ ID NO: 30 - DNA, 20 bp, Artificial Sequence
  Synthetic nucleotide
  misc_feature (10)..(11): fluorescent dye
ggagggacggggcaggagga

SEQ ID NO: 31 - DNA, 20 bp, Artificial Sequence
  Synthetic nucleotide
  misc_feature (11)..(12): fluorescence dye
aggagggacggggcaggagg

SEQ ID NO: 32 - DNA, 19 bp, Artificial Sequence
  Synthetic nucleotide
  misc_feature (9)..(10): fluorescence dye
agggacggggcaggaggag

SEQ ID NO: 33 - DNA, 19 bp, Artificial Sequence
  Synthetic nucleotide
  misc_feature (9)..(10): fluorescence dye
gagggacggggcaggagga

SEQ ID NO: 34 - DNA, 19 bp, Artificial Sequence
  Synthetic nucleotide
  misc_feature (10)..(11): fluorescence dye
ggagggacggggcaggagg

SEQ ID NO: 35 - DNA, 19 bp, Artificial Sequence
  Synthetic nucleotide
  misc_feature (11)..(12): fluorescence dye
aggagggacggggcaggag

SEQ ID NO: 36 - DNA, 18 bp, Artificial Sequence
  Synthetic nucleotide
  misc_feature (7)..(8): fluorescence dye
gggacggggcaggaggag

SEQ ID NO: 37 - DNA, 18 bp, Artificial Sequence
  Synthetic nucleotide
  misc_feature (8)..(9): fluorescence dye
agggacggggcaggagga

SEQ ID NO: 38 - DNA, 18 bp, Artificial Sequence
  Synthetic nucleotide
  misc_feature (9)..(10): fluorescence dye
gagggacggggcaggagg

SEQ ID NO: 39 - DNA, 18 bp, Artificial Sequence
  Synthetic nucleotide
  misc_feature (10)..(11): fluorescence dye
ggagggacggggcaggag

SEQ ID NO: 40 - DNA, 18 bp, Artificial Sequence
  Synthetic nucleotide
  misc_feature (11)..(12)
aggagggacggggcagga

SEQ ID NO: 41 - DNA, 17 bp, Artificial Sequence
  Synthetic nucleotide
  misc_feature (7)..(8): fluorescence dye
gggacggggcaggagga

SEQ ID NO: 42 - DNA, 17 bp, Artificial Sequence
  Synthetic nucleotide
  misc_feature (8)..(9): fluorescence dye
agggacggggcaggagg

SEQ ID NO: 43 - DNA, 17 bp, Artificial Sequence
  Synthetic nucleotide
  misc_feature (9)..(10): fluorescence dye
gagggacggggcaggag

SEQ ID NO: 44 - DNA, 17 bp, Artificial Sequence
  Synthetic nucleotide
  misc_feature (10)..(11): fluorescence dye
ggagggacggggcagga

SEQ ID NO: 45 - DNA, 16 bp, Artificial Sequence
  Synthetic nucleotide
  misc_feature (7)..(8): fluorescence dye
gggacggggcaggagg

SEQ ID NO: 46 - DNA, 16 bp, Artificial Sequence
  Synthetic nucleotide
  misc_feature (8)..(9): fluorescence dye
agggacggggcaggag

SEQ ID NO: 47 - DNA, 16 bp, Artificial Sequence
  Synthetic nucleotide
  misc_feature (9)..(10): fluorescence dye
gagggacggggcagga

SEQ ID NO: 48 - DNA, 15 bp, Artificial Sequence
  Synthetic nucleotide
  misc_feature (7)..(8): fluorescence dye
gggacggggcaggag

SEQ ID NO: 49 - DNA, 15 bp, Artificial Sequence
  Synthetic nucleotide
  misc_feature (8)..(9): fluorescence dye
agggacggggcagga

SEQ ID NO: 50 - DNA, 14 bp, Artificial Sequence
  Synthetic nucleotide
  misc_feature (7)..(8): fluorescence dye
gggacggggcagga

SEQ ID NO: 51 - DNA, 15 bp, Artificial Sequence
  Synthetic nucleotide
ggttggtgtggttgg

SEQ ID NO: 52 - DNA, 26 bp, Artificial Sequence
  Synthetic nucleotide
ggtggtggtggttgtggtggtggtgg

SEQ ID NO: 53 - DNA, 25 bp, Artificial Sequence
  Synthetic nucleotide
tgtgggggtggacgggccgggtaga

SEQ ID NO: 54 - DNA, 25 bp, Artificial Sequence
  Synthetic nucleotide
tgtgggggtggactgggtgggtacc

SEQ ID NO: 55 - DNA, 7 bp, Artificial Sequence
  Synthetic nucleotide
ttgggtt

SEQ ID NO: 56 - DNA, 31 bp, Artificial Sequence
  Synthetic nucleotide
gggggtgggagggtaggccttaggtttctga

SEQ ID NO: 57 - DNA, 30 bp, Artificial Sequence
  Synthetic nucleotide
ggtggtggggggggttggtagggtgtcttc

SEQ ID NO: 58 - DNA, 20 bp, Artificial Sequence
  Synthetic nucleotide
gggttagggttagggtaggg

SEQ ID NO: 59 - DNA, 25 bp, Artificial Sequence
  Synthetic nucleotide
cctgggggagtattgcggaggaagg

SEQ ID NO: 60 - RNA, 12 nt, Artificial Sequence
  Synthetic nucleotide
ggaggaggagga

SEQ ID NO: 61 - RNA, 6 nt, Artificial Sequence
  Synthetic nucleotide
ggagga

SEQ ID NO: 62 - DNA, 21 bp, Artificial Sequence
  Synthetic nucleotide
  misc_feature (1)..(1): 5Cy3 labelled
aaaaaaaaaaaaaaaaaaaaa

SEQ ID NO: 63 - DNA, 21 bp, Artificial Sequence
  Synthetic nucleotide
ctctctctctctctctctctc

SEQ ID NO: 64 - DNA, 21 bp, Artificial Sequence
  Synthetic nucleotide
  misc_feature (1)..(1): 5Cy3 labelled
ttttttttttttttttttttt

SEQ ID NO: 65 - DNA, 21 bp, Artificial Sequence
  Synthetic nucleotide
  misc_feature (1)..(1): 5Cy3 labelled
ccccccccccccccccccccc

SEQ ID NO: 66 - DNA, 21 bp, Artificial Sequence
  Synthetic nucleotide
  misc_feature (1)..(1): 5Cy3 labelled
tgaatgaatgactgcctgact

SEQ ID NO: 67 - DNA, 25 bp, Artificial Sequence
  Synthetic nucleotide
  misc_feature (1)..(1): 5Cy3 labelled
gaaaaagttaggactgctcgtcatc

SEQ ID NO: 68 - DNA, 21 bp, Artificial Sequence
  Synthetic nucleotide
  misc_feature (1)..(1): 5Cy3 labelled
gaaaagaatgactgcctgact

SEQ ID NO: 69 - DNA, 21 bp, Artificial Sequence
  Synthetic nucleotide
  misc_feature (1)..(1): 5Cy3 labelled
aagaagagtttactgtgaaga

SEQ ID NO: 70 - DNA, 21 bp, Artificial Sequence
  Synthetic nucleotide
  misc_feature (1)..(1): 5Cy3 labelled
cccccgaatgactgcctgact

SEQ ID NO: 71 - DNA, 21 bp, Artificial Sequence
  Synthetic nucleotide
  misc_feature (1)..(1): 5Cy3 labelled
tgcgcgaatgactgcctgact

SEQ ID NO: 72 - DNA, 21 bp, Artificial Sequence
  Synthetic nucleotide
  misc_feature (1)..(1): 5Cy3 labelled
taagttaggactgctcgtcat

SEQ ID NO: 73 - DNA, 22 bp, Artificial Sequence
  Synthetic nucleotide
  misc_feature (1)..(1): 5Cy3 labelled
taagttaggactgctcgtcatc

SEQ ID NO: 74 - DNA, 25 bp, Artificial Sequence
  Synthetic nucleotide
  misc_feature (1)..(1): 5Cy3 labelled
cccttagttaggactgctcgtcatc

SEQ ID NO: 75 - DNA, 21 bp, Artificial Sequence
  Synthetic nucleotide
  misc_feature (1)..(1): 5Cy3 Labelled
ttcttcagttcagccatccat

SEQ ID NO: 76 - DNA, 21 bp, Artificial Sequence
  Synthetic nucleotide
  misc_feature (1)..(1): 5Cy3 Labelled
gggaagcccggtgccgagaag

SEQ ID NO: 77 - DNA, 21 bp, Artificial Sequence
  Synthetic nucleotide
  misc_feature (1)..(1): 5Cy3 Labelled
ctcttcagttcagccatctca

SEQ ID NO: 78 - DNA, 21 bp, Artificial Sequence
  Synthetic nucleotide
  misc_feature (1)..(1): 5Cy3 Labelled
cccttgaatgactgcctgact

SEQ ID NO: 79 - DNA, 21 bp, Artificial Sequence
  Synthetic nucleotide
  misc_feature (1)..(1): 5Cy3 lablled
gagaagaatgactgcctgact

SEQ ID NO: 80 - DNA, 21 bp, Artificial Sequence
  Synthetic nucleotide
  misc_feature (1)..(1): 5Cy3 lablled
ggggggggggactgactgact

SEQ ID NO: 81 - DNA, 21 bp, Artificial Sequence
  Synthetic nucleotide
  misc_feature (21)..(21): 3Cy3 lablled
aaaaaaaaaaaaaaaaaaaaa

SEQ ID NO: 82 - DNA, 21 bp, Artificial Sequence
  Synthetic nucleotide
  misc_feature (21)..(21): 3Cy3 lablled
tgaatgaatgactgccttccc

SEQ ID NO: 83 - DNA, 21 bp, Artificial Sequence
  Synthetic nucleotide
  misc_feature (21)..(21): 3Cy3 lablled
tgaatgaatgactgccaatag

SEQ ID NO: 84 - DNA, 21 bp, Artificial Sequence
  Synthetic nucleotide
  misc_feature (21)..(21): 3Cy3 lablled
ctctctctctctctctctctc

SEQ ID NO: 85 - DNA, 21 bp, Artificial Sequence
  Synthetic nucleotide
  misc_feature (21)..(21): 3Cy3 lablled
tgaatgaatgactgccaaaag

SEQ ID NO: 86 - DNA, 21 bp, Artificial Sequence
  Synthetic nucleotide
  misc_feature (21)..(21): 3Cy3 lablled
tgaatgaatgactgccccccc

SEQ ID NO: 87 - DNA, 21 bp, Artificial Sequence
  Synthetic nucleotide
  misc_feature (21)..(21): 3Cy3 lablled
ccccccccccccccccccccc

SEQ ID NO: 88 - DNA, 21 bp, Artificial Sequence
  Synthetic nucleotide
  misc_feature (21)..(21): 3Cy3 lablled
ttttttttttttttttttttt

SEQ ID NO: 89 - DNA, 21 bp, Artificial Sequence
  Synthetic nucleotide
  misc_feature (21)..(21): 3Cy3 lablled
tgaatgaatgactgcctgact

SEQ ID NO: 90 - DNA, 21 bp, Artificial Sequence
  Synthetic nucleotide
  misc_feature (21)..(21): 3Cy3 lablled
ttcttcagttcagccatccat

SEQ ID NO: 91 - DNA, 21 bp, Artificial Sequence
  Synthetic nucleotide
  misc_feature (21)..(21): 3Cy3 lablled
gagaagcccggtgccgagaag

SEQ ID NO: 92 - DNA, 21 bp, Artificial Sequence
  Synthetic nucleotide
  misc_feature (21)..(21): 3Cy3 lablled
tgaatgaatgactgccaagag

SEQ ID NO: 93 - DNA, 21 bp, Artificial Sequence
  Synthetic nucleotide
  misc_feature (21)..(21): 3Cy3 lablled
actgactgactgggggggggg

SEQ ID NO: 94 - DNA, 21 bp, Artificial Sequence
  Synthetic nucleotide
  misc_feature (21)..(21): 3Cy3 lablled
aagaagagtttactgtgaaga

SEQ ID NO: 95 - DNA, 21 bp, Artificial Sequence
  Synthetic nucleotide
  misc_feature (21)..(21): 3Cy3 lablled
taagttaggactgctcgtcat

SEQ ID NO: 96 - DNA, 22 bp, Artificial Sequence
  Synthetic nucleotide
  misc_feature (11)..(12): Internal iCy3
aaaaaaaaaaaaaaaaaaaaaa

SEQ ID NO: 97 - DNA, 22 bp, Artificial Sequence
  Synthetic nucleotide
  misc_feature (11)..(12): Internal iCy3
tttttttttttttttttttttt

SEQ ID NO: 98 - DNA, 19 bp, Artificial Sequence
  Synthetic nucleotide
  misc_feature (9)..(10): Internal iCy3
ccctgataaaaagcctacc

SEQ ID NO: 99 - DNA, 20 bp, Artificial Sequence
  Synthetic nucleotide
  misc_feature (4)..(5): Internal iCy3
tgaagaatgactgcctgact

SEQ ID NO: 100 - DNA, 22 bp, Artificial Sequence
  Synthetic nucleotide
  misc_feature (11)..(12): Internal iCy3
cccccccccccccccccccccc

SEQ ID NO: 101 - DNA, 20 bp, Artificial Sequence
  Synthetic nucleotide
  misc_feature (8)..(9)
tgaatgaagactgcctgact

SEQ ID NO: 102 - DNA, 20 bp, Artificial Sequence
  Synthetic nucleotide
  misc_feature (9)..(10): Internal iCy3
gaaaaccgtacttccaatcc

SEQ ID NO: 103 - DNA, 20 bp, Artificial Sequence
  Synthetic nucleotide
  misc_feature (12)..(13): Internal iCy3
tgaatgaatgacgcctgact

SEQ ID NO: 104 - DNA, 20 bp, Artificial Sequence
  Synthetic nucleotide
  misc_feature (13)..(14): Internal iCy3
accgaaggaacattcgtccg

SEQ ID NO: 105 - DNA, 20 bp, Artificial Sequence
  Synthetic nucleotide
  misc_feature (11)..(12): Internal iCy3
ttgatgagcccaggaagttg

SEQ ID NO: 106 - DNA, 20 bp, Artificial Sequence
  Synthetic nucleotide
  misc_feature (16)..(17): Internal iCy3
tgaatgaatgactgccgact

SEQ ID NO: 107 - DNA, 22 bp, Artificial Sequence
  Synthetic nucleotide
  misc_feature (11)..(12): Internal iCy3
aggagggacggggcaggaggag

SEQ ID NO: 108 - DNA, 18 bp, Artificial Sequence
  Synthetic nucleotide
  misc_feature (9)..(10): Internal iCy3
gagggacggggcaggagg

SEQ ID NO: 109 - DNA, 18 bp, Artificial Sequence
  Synthetic nucleotide
  misc_feature (9)..(10): Internal iCy5
gagggacggggcaggagg

SEQ ID NO: 110 - DNA, 19 bp, Artificial Sequence
  Synthetic nucleotide
gagggacggtggcaggagg